(19) United States
(12) Patent Application Publication
(10) Pub. No.: US 2023/0416851 A1
(43) Pub. Date: Dec. 28, 2023

(54) RAPID DIAGNOSTIC ELECTROCHEMICAL BIOSENSING TARGETED WITH ANTISENSE OLIGONUCLEOTIDES

(71) Applicants: University of Maryland, Baltimore, Baltimore, MD (US); University of Maryland, Baltimore County, Baltimore, MD (US)

(72) Inventors: Dipanjan PAN, Ellicott City, MD (US); Maha Mohammad Shehadeh ALAFEEF, Baltimore, MD (US); Ketan DIGHE, Ellicott City, MD (US); Parikshit MOITRA, Baltimore, MD (US)

(73) Assignees: University of Maryland, Baltimore, Baltimore, MD (US); University of Maryland, Baltimore County, Baltimore, MD (US)

(21) Appl. No.: 18/247,474

(22) PCT Filed: Oct. 5, 2021
(86) PCT No.: PCT/US21/53575

§ 371 Date: Mar. 31, 2023

Related U.S. Application Data
(60) Provisional application No. 63/181,599, filed on Apr. 29, 2021.
(60) Provisional application No. 63/163,203, filed on Mar. 19, 2021.
(60) Provisional application No. 63/120,809, filed on Dec. 3, 2020.
(60) Provisional application No. 63/106,916, filed on Oct. 29, 2020.
(60) Provisional application No. 63/088,179, filed on Oct. 6, 2020.

(51) Int. Cl. C12Q 1/70; C12Q 1/6825; B01L 3/00

(52) U.S. Cl. CPC C12Q 1/701; C12Q 1/6825; B01L 3/502715; B01L 2300/0663; B01L 2200/16

(57) ABSTRACT

The present disclosure relates to electrochemical biosensing systems and methods that can be adapted to accurately and rapidly detect a target gene in clinical samples, using anti-sense oligonucleotides for selective detection of biological pathogens.

### CROSS REFERENCE TO RELATED APPLICATIONS

This application claims the benefit of and priority from U.S. Provisional Application No. 63/088,179 filed on Oct. 6, 2020, U.S. Provisional Application No. 63/106,916 filed Oct. 29, 2020, U.S. Provisional Application No. 63/120,809 filed on Dec. 3, 2020, U.S. Provisional Application No. 63/163,203 filed on Mar. 19, 2021 and U.S. Provisional Application 63/181,599 filed on Apr. 29, 2021, the entire contents of which are fully incorporated herein by reference.

### GOVERNMENT SUPPORT

This invention was made with government support under Grant Number EB028026 awarded by The National Institutes of Health. The government has certain rights in the invention.

### FIELD OF THE INVENTION

The invention relates to systems and methods for the detection of biological pathogens. In particular, the invention relates to the detection of targeted nucleic acid sequences using an electrochemical biosensor with targeted antisense oligonucleotides.

### BACKGROUND OF THE INVENTION

Rapid identification of specific nucleic acid sequences has enormous potential in disease diagnosis and management. One of the most common applications of nucleic acid detection is molecular-based diagnostic tests or nucleic acid testing for the diagnosis of various infectious diseases caused by different microbes and pathogens.

Nucleic acid testing (NAT) or nucleic acid amplification testing (NAAT) is a process that involves amplification and identification of nucleic acids for diagnosis and/or guidance of therapy. Commercially available nucleic acid detection methodologies typically involve amplification of nucleic acid extracted from the bio-fluids collected from the patients under observation.

Globally, infectious diseases have an enormous impact on the community from a social as well as economic standpoint. For example, according to the World Health Organization, as of September 2021 there have been more than 220 million confirmed cases worldwide of severe acute respiratory syndrome coronavirus 2 (SARS-CoV-2), the virus that is responsible for the COVID-19 pandemic, leading to more than 4.5 million deaths and substantial socio-economic disruption.

A crucial shortcoming of the healthcare systems across the globe has been the ability to rapidly and accurately diagnose the disease, with contributing factors that include a shortage of test kits and specimen materials, availability of personal protective equipment and reagents, and limited certified testing centers. Further, the lack of rapid diagnostic tests along with the inaccessibility of the advanced instrumental techniques to all the diagnostic centers, especially the remote ones, contributes to the confusion surrounding which individuals should be quarantined, limits epidemiological data, and hinders tracking of pathogen transmission within as well as across communities.

The ability to perform pervasive testing has already shown benefits to countries such as South Korea and Singapore, providing precise information about mandatory quarantine for a carrier of the virus and rigorous contact tracing which in turn results in greater control in slowing the spread of the disease. Downmodulating the infection rate will therefore help to minimize the risk of overwhelming health facilities.

Current standard practice for detecting an active COVID-19 infection uses chest X-ray, chest CT, or reverse transcriptase real-time polymerase chain reaction (RT-PCR) which requires labor-intensive, laboratory-based protocols for lysis, viral RNA isolation, and removal of inhibiting materials. On the basis of this technique, numerous laboratories have developed experimental protocols using quantitative RT-PCR (qRT-PCR) methods for virus identification within 4 to 6 hours, including a test developed by U.S. Centers for Disease Control and Prevention (CDC) and approved under emergency use authorization (EUA) process. However, previously described testing protocols using RT-PCR often cannot report positive COVID-19 cases at its initial presentation, due to limitations of sample collection and transportation. Furthermore, traveling to a clinical setting for testing increases the risk of spreading the SARS-CoV-2 virus which further adds strain to a resource-limited healthcare system. These restrictions become major hurdles for situations with resource scarcity.

Additionally, while serological tests are rapid, point-of-care (POC), and require minimal equipment, their efficacy may be limited in the diagnosis of acute SARS-CoV-2 infection, as it may take several days to weeks after the onset of the symptom for a patient to develop a detectable antibody response.

Further, several variants of the SARS-CoV-2 virus have proved particularly concerning, because they have been observed to be more readily transmissible, and public health officials are concerned that vaccines may not be fully effective against such variants. Variants have resulted in additional lockdowns within many countries and restrictions on international travel, in attempts to curb the spread of variants.

Therefore, there is an urgent need for other approaches that are low-cost, rapid and provide diagnosis at the POC level. Therefore, new solutions and methodologies for nucleic acid detection are in high demand.

The COVID-19 pandemic has demonstrated there is a need for tests for the identification of biological pathogens in samples, particularly for tests which (i) do not need prior extraction and amplification steps to be performed; (ii) do not demand the use of advanced equipment (e.g., centrifuge, thermocycler, etc.); (iii) use low-cost and easily accessible materials that can be rapidly manufactured in bulk; (iv) exhibit a high degree of selectivity and sensitivity and/or (v) have a short turnaround time.

While the need for tests to rapidly, selectively, and efficiently identify and detect biological pathogens in samples has been aptly demonstrated in the COVID-19 pandemic, the need for such tests goes beyond the detection of SARS-CoV-2. There is a need for improved tests to detect biological pathogens across many therapeutic categories, including but not limited to detection of biological pathogens which are related to pandemic disease (including for instance the presence of biological pathogens related to outbreaks of COVID-19, SARS, MERS, Ebola, or Bird Flu), biological pathogens related to respiratory disease (including for instance influenza A or B, Streptococcus, tonsillitis, pharyngitis, or adenovirus), biological pathogens related to sexually transmitted infections (including herpes, gonorrhea, syphilis, or chlamydia), and biological pathogens related to gynecological infections (including HPV, UTI, bacterial vaginosis, and trichomonas).

Therefore, as some of the existing techniques remain laborious and technically challenging, there is an urgent unmet need for a rapid, cost-effective, and selective diagnostic test for biological pathogens that can provide fast and accurate test results within minutes.

### SUMMARY OF THE INVENTION

Disclosed herein are systems and methods for an electrochemical biosensing system to detect biological pathogens.

In one embodiment, there is provided an electrochemical biosensor for use in the detection of a biological pathogen in a sample, the electrochemical biosensor comprising: a sensing element comprising a plurality of first anti-sense oligonucleotides, the sequence of which is complementary to a first nucleic acid sequence in a target gene of the biological pathogen; a first electrode connected to a first end of each of the plurality of anti-sense oligonucleotides; and a second electrode electrically connected to the first electrode, wherein contact of the sample with the first electrode causes binding of the plurality of first anti-sense oligonucleotides to the first nucleic acid sequence in the target gene, to provide a signal to identify presence of the biological pathogen.

In another embodiment, the electrochemical biosensor further comprises a plurality of second anti-sense oligonucleotides, the sequence of which is complementary to a second nucleic acid sequence in the target gene of the biological pathogen near to the first nucleic acid sequence, wherein the first electrode is additionally connected to a first end of the plurality of second anti-sense oligonucleotides, and wherein the signal is provided additionally by binding of the plurality of second anti-sense oligonucleotides to the second nucleic acid sequence in the target gene.

In another embodiment, the biological pathogen is SARS-CoV-2. In another embodiment, the sequence of the first anti-sense oligonucleotide comprises SEQ ID NO 6, SEQ ID NO 7, SEQ ID NO 8, or SEQ ID NO 9. In another embodiment, the sequence of the second anti-sense oligonucleotide comprises SEQ ID NO 6, SEQ ID NO 7, SEQ ID NO 8, or SEQ ID NO 9. In another embodiment, the first anti-sense oligonucleotide has an unpaired probability for the first nucleic acid sequence of at least 0.5. In another embodiment, the first anti-sense oligonucleotide has a binding energy of less than −8 kcal/mol. In another embodiment, the first anti-sense oligonucleotide has a tendency to form a hairpin-loop structure.

In another embodiment, the second electrode of the electrochemical biosensor is a counter electrode or a reference electrode. In another embodiment, the electrochemical biosensor further comprises a third electrode electrically connected to the first and second electrodes. In another embodiment, the third electrode is a counter electrode or a reference electrode.

In another embodiment, the electrochemical biosensor further comprises a substrate and a conductive film coated on the surface of the substrate, wherein the first and second electrodes are deposited on the conductive film. In another embodiment, the first anti-sense oligonucleotides are capped with conductive nanoparticles. In another embodiment, the conductive nanoparticles are gold nanoparticles. In another embodiment, the conductive film comprises graphene.

In one embodiment, the first anti-sense oligonucleotides of the electrochemical biosensor form hairpin-loop structures in the absence of the target gene, and the presence of the target gene in the sample causes the plurality of first anti-sense oligonucleotides to unfold and bind to the first nucleic acid sequences in the target gene, resulting in providing the signal. In another embodiment, the electrochemical biosensor further comprises a redox reporter molecule bound to the second end of each of the plurality of first anti-sense oligonucleotides, such that when the first anti-sense oligonucleotide forms a hairpin-loop structure, the redox reporter molecule is brought within proximity of the first electrode, and wherein in the presence of the target gene the first anti-sense oligonucleotide unfolds moving the redox reporter molecule away from the first electrode, resulting in providing the signal. In one embodiment, the redox reporter molecule is methylene blue.

In one embodiment, there is provided a method of detecting a biological pathogen in a sample, the method comprising: providing a plurality of first anti-sense oligonucleotides having a sequence complementary to a first nucleic acid sequence in a target gene of the biological pathogen; providing a first electrode and a second electrode electrically connected to one another; connecting a first end of the pluralities of the first anti-sense oligonucleotides to the first electrode; contacting the first electrode to the sample; and measuring a signal from the first and second electrodes, wherein the binding of the target gene of the biological pathogen to the plurality of first anti-sense oligonucleotides provides the signal identifying the presence of the biological pathogen in the sample. In another embodiment, the method further comprises: providing a plurality of second anti-sense oligonucleotides, the sequence of which is complementary to a second nucleic acid sequence in the target gene of the biological pathogen near to the second nucleic acid sequence; and connecting a first end of the pluralities of the second anti-sense oligonucleotides to the first electrode, wherein the signal is provided additionally by binding of the target gene of the biological pathogen to the plurality of second anti-sense oligonucleotides.

In one embodiment, there is provided a method of selecting at least one anti-sense oligonucleotide probe for use in detection of a biological pathogen, the method comprising: (a) identifying a target gene in the biological pathogen; (b) obtaining the nucleic acid sequence of the target gene; (c) producing a library of anti-sense oligonucleotides of a length of about 20 nucleotides wherein the sequence of each anti-sense oligonucleotide is complementary to a section of the nucleic acid sequence in the target gene and wherein: (i) guanine and cysteine form from 40 to 60 percent of each anti-sense oligonucleotide in the library; (ii) none of the anti-sense oligonucleotides in the library are complementary to a section of the target gene with the sequence GGGG; and (iii) the average unpaired probability of each of the anti-sense oligonucleotides in the library is at least 0.5; (d) ranking the anti-sense oligonucleotides in the library in descending order of average unpaired probability; and (e) selecting the at least one anti-sense oligonucleotide probe from the anti-sense oligonucleotides in the library, wherein the binding energy of the at least one anti-sense oligonucleotide probe is less than −8 kcal/mol, compared with the nucleic acid sequence of the target gene. In another embodiment, the at least one anti-sense oligonucleotide probe is additionally selected based on comparative binding disruption energies and binding energies with the nucleic acid sequence of the target gene. In another embodiment, the at least one anti-sense oligonucleotide is additionally selected for a tendency to form a hairpin-loop structure.

### BRIEF DESCRIPTION OF THE DRAWINGS

FIG. 1 shows a schematic representation of the electrochemical biosensing system according to non-limiting embodiments.

FIG. 2 shows the average response of the electrochemical biosensor according to non-limiting embodiments in the frequency range of 50 kHz to 6911.98 Hz normalized to the blank signal (water) when tested with direct clinical samples treated with lysis buffer with no RNA extraction step.

FIG. 3 shows the UV-Vis absorbance spectrum of the citrate stabilized gold nanoparticle before and after capping with the thiol modified antisense oligonucleotide probes, Px (ASOs).

FIG. 4 shows a comparative agarose gel electrophoretic mobility of the gold nanoparticles stabilized by citrate and ssDNA probes.

FIG. 5A shows a schematic representation of the proposed concept behind the enhancement in electrochemical response using gold nanoparticles, when capped with ASOs, in accordance with non-limiting embodiments. FIG. 5B shows the relative change in the biosensor output voltage for two sensor configurations, according to non-limiting embodiments. FIG. 5C shows comparative biosensor signal output for both sensor configurations, according to non-limiting embodiments.

FIG. 6A shows sensor output signal as a function of time with the addition of SARS-CoV-2 viral RNA load (5.85×10⁴ copies/μL). FIG. 6B shows the standard curve obtained from the electrochemical sensor with an increasing concentration of SARS-CoV-2 viral RNA. FIG. 6C shows comparative electrochemical responses in the presence of SARS-CoV-2 RNA obtained from the pristine sensor, the pristine sensor when deposited with citrate-stabilized gold nanoparticles (AuNPs), and ASO-capped AuNPs (Au probe). FIG. 6D shows the relative electrochemical response of the sensor to MERS-CoV and SARS-CoV RNA when compared to SARS-CoV-2 viral RNA.

FIG. 7 shows a schematic representation of the operation principle of an electrochemical biosensor according to non-limiting embodiments, wherein step A includes collecting the infected samples from the nasal swab or saliva of the patients under observation, step B includes extracting the viral RNA, step C includes adding the viral RNA on top of a graphene-ASO-AuNP platform, step D includes an incubation period of 5 minutes and step E includes recording the digital electrochemical output.

FIG. 8 shows a thiol-modified antisense oligonucleotide conjugated to methylene blue with a hairpin loop structure, according to non-limiting embodiments.

FIG. 9 shows a thiol-modified antisense oligonucleotide conjugated to methylene blue with a hairpin loop structure, according to other non-limiting embodiments.

FIG. 10 shows the results of using an electrochemical biosensor having an ASO with a hairpin loop structure conjugated to methylene blue for the detection of SARS-CoV-2, according to non-limiting embodiments.

FIG. 11A shows the baseline cyclic voltammetry curve observed before and after sensor conjugation with methylene blue attached antisense oligonucleotides in lysis buffer, according to non-limiting embodiments. FIG. 11B shows a higher change in current for positive samples compared to baseline. FIG. 11C shows the change in current post addition of COVID-19 positive sample to the sensor due to increased distance between methylene blue and sensor electrodes because of selective ASO-RNA.

FIG. 12 shows the average response of the electrochemical biosensor according to non-limiting embodiments in the frequency range of 455119 Hz-597182 Hz normalized to the blank signal (Water) when tested with RNA extracted from clinical samples.

FIG. 13 shows differentially functionalized ASOs with their sequences, according to non-limiting embodiments.

FIG. 14A shows a design principle of antisense oligonucleotides with differential functionalized ends, according to non-limiting embodiments, wherein the yellow dot represents the ASO-capped gold nanoparticles. FIG. 14B shows the backbone 3D structure of ASO1 (P1) and FIG. 14C shows the backbone 3D structure of ASO2 (P2) according to non-limiting embodiments. FIG. 14D shows differentially functionalized AuNPs with thiol-ended ASOs along with their sequences, according to non-limiting embodiments. FIG. 14E shows comparative target N-gene binding and binding site disruption energies of the four selected ASOs, according to non-limiting embodiments.

FIG. 15A shows a zoom-in image illustrating the layer-by-layer construct of an electrochemical biosensor with filter paper, a graphene film and AuNP according to non-limiting embodiments. FIG. 15B shows a dark-field image of the graphene film, as well as a photograph of the graphene sensor platform. FIG. 15C shows SEM images of the graphene-based platform.

FIG. 16 shows a I-V curve of graphene nanoplatelet coated filter paper for different concentrations, with the insert representing the I-V curve at 5 mg/ml.

FIG. 17 shows the optimization of ASOs capped with AuNPs at different ratios to achieve maximum sensitivity and optimal signal output, according to non-limiting embodiments.

FIG. 18 shows the changes in hydrodynamic diameter of the ASO-capped gold nanoparticles when added with SARS-CoV-2 viral RNA (1 ng/mL, i.e., 5.854*10⁷ copies per mL), in accordance with non-limiting embodiments. The hydrodynamic diameters were shown at three different concentrations for (a) probe 1; (b) probe 2; (c) probe 3 and (d) probe 4.

FIG. 19 shows the comparative change in hydrodynamic diameter of the ASO-capped gold nanoparticles after the addition of SARS-CoV-2 viral RNA (1 ng/mL, i.e., 5.854*10⁷ copies per mL), in accordance with non-limiting embodiments.

FIG. 20 shows the sensor output response with varying sizes of functionalized spherical gold nanoparticles, in accordance with non-limiting embodiments.

FIG. 21 shows a UV-Visible spectrum of Au-Pₘᵢₓ in the absence and presence of SARS-CoV-2 viral RNA, according to non-limiting embodiments.

FIG. 22 shows the sensor output signal to understand the distribution of the probe coverage on the surface of the electrodes, according to non-limiting embodiments.

FIGS. 23A to 23F show the following SEM images. FIG. 23A shows a sensor without any addition of sample. FIG. 23B shows sample addition with a volume of 10 μL. FIG. 23C shows sample addition with a total volume of 12 μL. A zoom-in image of FIG. 23C is shown in FIG. 23D, FIG. 23E and FIG. 23F.

FIG. 24 shows a SEM image of the Whatman filter paper indicating the fibrous structures.

FIG. 25A shows the real-time response of the sensor chip toward COVID-19 positive clinical samples and healthy samples. FIG. 25B shows a comparison of the sensor chip response among a representative set of clinical samples from 15 healthy asymptomatic samples and 15 confirmed COVID-19 positive samples. FIG. 25C shows a schematic diagram for the sources of clinical samples that have been collected either from a nasopharyngeal swab or saliva. FIG. 25D shows an analysis and comparison with the actual RNA concentration for three clinical samples. The sensor response toward each clinical sample has been recorded in three repetitive measurements. The average of three measurements has been used to estimate the RNA concentration using the standard curve shown in FIG. 25E, r²=0.999, obtained from standard SARS-CoV-2 genomic RNA samples.

FIG. 26A shows a summary of the results of classifying 48 clinical samples into COVID-19 positive or negative cases using the developed sensor chip, which was benchmarked to the standard SARS-CoV-2 diagnostic kit (LabGun COVID-19 RT-PCR kit). FIG. 26B shows a confusion matrix of the classification results obtained by applying thresholding (threshold=0.13) on the sensor chip response against the 48 clinical samples.

FIG. 27 shows an electrochemical biosensor device in accordance with non-limiting embodiments.

### DETAILED DESCRIPTION OF THE INVENTION

This disclosure, and the embodiments described herein, are provided by way of illustration of an example, or examples, of particular embodiments of the principles of the present invention. These examples are provided for the purposes of explanation, and not limitation, of those principles and of the invention. It will be appreciated that numerous specific details have been provided for a thorough understanding of the exemplary embodiments described herein. However, it will be understood by those of ordinary skill in the art that the embodiments described herein may be practiced without these specific details. In other instances, well-known methods, procedures, apparatus, equipment and components have not been described in detail so as not to obscure the embodiments described herein. Furthermore, this description is not to be considered so that it may limit the scope of the embodiments described herein in any way, but rather as merely describing the implementation of the various embodiments described herein.

### Definitions

The terminology used herein is for the purpose of describing particular embodiments only and is not intended to be limiting of example embodiments of the invention. Terms and phrases used in this specification have their ordinary meanings as one of skill in the art would understand.

As used herein, “ASO” means anti-sense oligonucleotide. “Thiol-modified” means an oligonucleotide which has been functionalized to have a thiol moiety at either its 5′ or 3′ end. “Nanoparticles” means a particle of matter less than 500 nanometers in diameter. “AuNP” means gold nanoparticles. “Capped” nanoparticle means a nanoparticle which is covalently bound to another molecule, such as a thiol-modified ASO.

As used herein, a “biological pathogen” may include any bacteria, virus, fungus, or other organism which is capable of causing disease or deleterious health effects in a human or animal subject. As used herein, a “clinical sample” or “sample” may include any sample, including a fluid or tissue sample, taken from a subject. As used herein, a “positive sample” is a sample that either contains a biological pathogen of interest or contains a concentration of a biological pathogen of interest above a defined threshold. As used herein, a “negative sample” is a sample that either does not contain a biological pathogen of interest or contains a biological pathogen of interest at a concentration below a defined threshold.

### Electrochemical Biosensor

The present disclosure relates to nanotechnology-based electrochemical biosensors, systems and methods that can be adapted to accurately detect a target gene in clinical samples, using anti-sense oligonucleotides (ASOs) for selective detection of biological pathogens. ASOs are designed to target specific gene segments of biological pathogens of interest and are functionalized and connected with at least one electrode. Extraction and amplification of nucleic acid is not a requirement, allowing the sensing of biological pathogens directly from clinical samples.

There is a need to develop detection approaches that are low-cost, rapid, do not require the use of advanced equipment. For instance, there is an immediate need for detection systems which can be used as a screening tool for the diagnosis of COVID-19 infection at point-of-care (POC). Rapid, low-cost and user-friendly molecular diagnostic methods are important for combating outbreaks of infectious diseases. Especially during the current pandemic of COVID-19, it is critical to expanding the testing capacity beyond laboratory settings. There is an immediate need to develop global testing capability over existing conventional approaches. In recognition of this unmet need, the World Health Organization (WHO) has established the ASSURED criteria (Affordable, Sensitive, Specific, User-friendly, Rapid and Robust, Equipment-free, and Deliverable)—as guidelines for tests to be effective in resource-limited environments.

In certain embodiments, the nanotechnology-based electrochemical biosensor described herein provides one or more advantages over the alternative currently available molecular techniques. In certain embodiments, the nanotechnology-based electrochemical sensing system described herein: (i) does not need prior nucleic acid extraction or amplification; (ii) does not demand the use of specialized chemicals, reagents, complex fabrication procedures or advanced equipment (e.g., centrifuge, thermocycler, etc.); (iii) may be used with low-cost and easily accessible materials that can be rapidly manufactured in bulk for commercialization; (iv) has a high degree of sensitivity and selectivity; (v) has a short turnaround time; (vi) is portable; (vii) may be operated solely at room or ambient temperatures and/or (viii) relies on quantitative measurements and data.

One advantage of certain embodiments of the presently described electrochemical biosensor is that the necessary materials are readily accessible (including in bulk) and relatively inexpensive, allowing for a low cost per test. For instance, certain embodiments of the system may be used to test samples without extraction of nucleic acids, thus avoiding the use of expensive commercial kits containing several steps and specialized laboratory equipment such as centrifuges. In certain embodiments the antisense oligonucleotides are short ssDNA sequences that are relatively economical to synthesize. Other materials used in various embodiments are relatively inexpensive and easy to obtain or synthesize on a large scale, such as gold nanoparticles, substrates, conductive films and redox reporters.

Another advantage of certain embodiments of the presently described electrochemical biosensor is that it may be used to test samples without requiring the amplification of nucleic acids. This means that almost no reagents are required to operate the electrochemical biosensor. It also eliminates the need to raise the temperature of the test sample above room temperature, which is a practically difficult and inconvenient step that is required for the operation of many tests known in the art.

In certain embodiments, the presently described system provides for rapid turnaround time for detection of a biological pathogen. In certain embodiments, once suitable ASOs have been designed and chosen as described herein, the detection of a biological pathogen of interest may be performed in less than five minutes, and in some embodiments within 2 or 3 minutes.

In certain embodiments, the presently described electrochemical biosensor may have a lower limit of detection compared with other conventional methods.

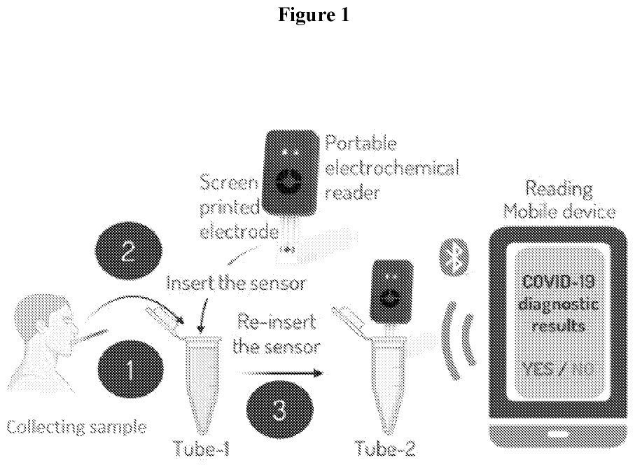

In certain embodiments, the electrochemical biosensor may be integrated into a portable device that can be used at point-of-care. Moreover, in certain embodiments, the device may be integrated with smartphones and other mobile devices for the easy and rapid detection of biological pathogens, as shown in FIG. 1.

The electrochemical biosensor disclosed herein may be adapted to detect target genes of various biological pathogens of interest. In some embodiments, this detection may be performed directly in clinical samples. For example, the system may be adapted to detect biological pathogens which are related to pandemic disease, including for instance biological pathogens related to outbreaks of COVID-19, SARS, MERS, Ebola, Zika virus, yellow fever, influenza, H1N1, polio, HIV or Bird Flu. The system may be adapted to detect biological pathogens related to respiratory disease, including for instance influenza A or B, Streptococcus, or adenovirus, or pathogens related to tonsillitis or pharyngitis. The system may be adapted to detect biological pathogens related to sexually transmitted infections, including herpes, gonorrhea, syphilis, or chlamydia, or biological pathogens related to gynecological infections including HPV, UTI, bacterial vaginosis, and trichomonas.

According to certain embodiments, the biosensor is configured to determine a sample measurement. In some embodiments, determining a sample measurement comprises determining an electrical characteristic of the sample. According to certain embodiments, the biosensor is configured to determine a reference measurement. In some embodiments, determining a reference measurement comprises determining an electrical characteristic of a reference. In some embodiments, the reference may include water, phosphate-buffered saline (PBS), buffer or the like. In some embodiments, the biosensor is configured to determine a reference measurement in a collection tube of water. In some embodiments, determining a sample measurement includes washing the biosensor with PBS either before or after taking the sample measurement.

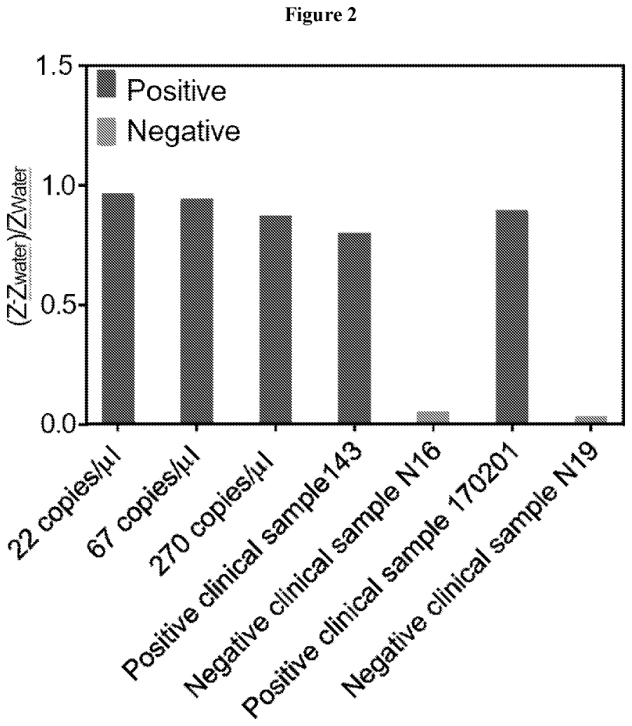

According to some embodiments, the electrochemical biosensor disclosed herein can differentiate between positive samples and negative samples, for example, as shown in FIG. 2. In certain embodiments, a positive sample is a sample that contains a biological pathogen of interest. In other embodiments, a positive sample is a sample that contains a concentration of a biological pathogen of interest above a defined threshold. In certain embodiments, a negative sample is a sample that does not contain a biological pathogen of interest. In other embodiments, a negative sample is a sample that contains a biological pathogen of interest at a concentration below a defined threshold. The defined threshold may be determined based on a variety of factors, including but not limited to the nature of the biological pathogen, the type and purpose of the test and the local environmental and public conditions.

According to some embodiments, the electrochemical biosensor can detect a biological pathogen of interest without signal cross talk with a detection limit around 2 copies/μL and a response time of around 2-3 minutes. Moreover, in some embodiments, the electrochemical biosensor offers a sensitivity of >98% and a specificity of 100% at standard laboratory testing conditions with clinical samples. In some embodiments, devices using the electrochemical biosensor disclosed herein can be configured to increase speed, sensitivity, specificity, portability, and affordability compared to typical test devices known in the art.

The electrochemical biosensor disclosed herein is configured to determine the presence of a biological pathogen by determining at least one electrical characteristic when the electrochemical biosensor is exposed to a sample. Advantageously, in some embodiments, the electrochemical biosensor may be configured for quantitative measurement of a biological pathogen in a sample without requiring nucleic acid extraction or amplification. In certain embodiments, the biosensor is configured to determine electrical characteristics of a sample. In some embodiments, the biosensor can be configured to measure resistance, conductivity, voltage, current or impedance. In one embodiment, the biosensor is configured to measure impedance of a sample. For example, the biosensor can be configured to measure impedance of a sample between a first electrode, such as a sensing electrode, and a second electrode, such as a reference or counter electrode. In some embodiments, the biosensor may be electrically coupled to a potentiometer, galvanometer, electrochemical impedance spectroscope, or other measuring devices known in the art.

The electrochemical biosensor disclosed herein comprises at least one sensing element and at least two electrodes. The electrochemical biosensor disclosed herein is configured to determine at least one electrical characteristic when the electrochemical biosensor is exposed to a sample.

### Sensing Element

The sensing element of the electrochemical biosensor, which may also be referred to as a biological receptor, is configured to detect the presence of specific biological pathogens in a sample. The sensing element of the electrochemical biosensor comprises at least one ASO. In certain embodiments, the sensing element of the electrochemical biosensor may comprise multiple ASOs. ASOs may be designed to target at least one gene of interest. In certain embodiments, the target gene may be present as ssDNA, dsDNA, or RNA.

Suitable ASOs are selected based on their target binding energy and disruption energy. In certain embodiments, pairs of ASOs may be chosen that bind to closely spaced regions in the target sequences. ASOs are functionalized at either the 5′ or 3′ end, such that they are capable of connecting to at least one electrode. In certain embodiments, ASOs are functionalized with a thiol moiety or carboxylic acid at either at the 5′ or 3′ end, such that they are capable of covalently binding to at least one electrode. In other embodiments, ASOs are functionalized with a thiol moiety or carboxylic either at the 5′ or 3′ end and capped with gold nanoparticles, which may then be coupled with at least one electrode. In some embodiments, at least one end of the ASOs is connected to at least one electrode. In some embodiments, the ASOs are conjugated to at least one electrode.

In certain embodiments, ASOs of a preferred length of 20 nucleotide bases are chosen. In one embodiment, ASOs are chosen using software for statistical folding of nucleic acids. In one such embodiment, the following criteria are used: (i) guanine and cysteine percent content (GC %) within 40-60%; (ii) target sequences with GGGG are excluded; (iii) average unpaired probability of the ASOs for target site nucleotides is set to ≥0.5; (iv) all sites targeted to the peak in the accessibility profile are ranked by their average unpaired probability (the higher the better), with the threshold probability above 0.5; (v) the top 20 candidate ASOs with the highest average unpaired probability are selected for further consideration; and (vi) the binding energy of the ASOs is compared with the target sequence and the binding energy cutoff for the selection of ASOs is set at ≤−8 kcal/mol. From candidate ASOs, at least one ASO is chosen based on comparative binding disruption energies and binding energies with the target sequence.

In one embodiment, multiple ASOs or multiple such pairs of ASOs are chosen, covering multiple regions of a target gene at the same time. One potential advantage of using multiple ASOs, covering multiple regions of a target gene, is accurate recognition of a target sequence even if other genomic segments of the biological pathogen are subject to mutation. In one embodiment, two such pairs of ASOs are chosen, covering two regions of a target gene at the same time.

In one embodiment, ASOs are chosen in pairs binding to closely spaced regions in the target sequence, and each ASO in such a pair is differentially functionalized such that the functionalized ends come close to each other when the two ASOs are bound to the target sequence. In some embodiments, the aggregation of the ASOs may enhance the electrochemical response.

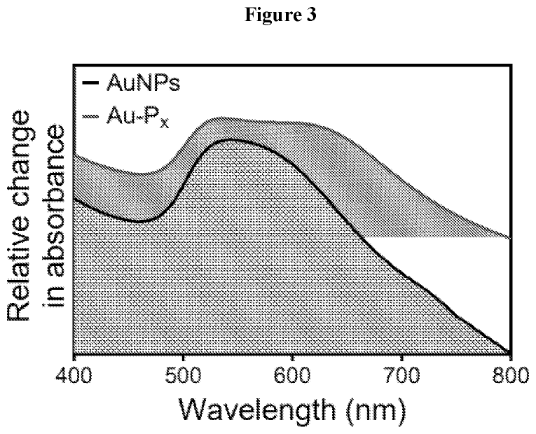

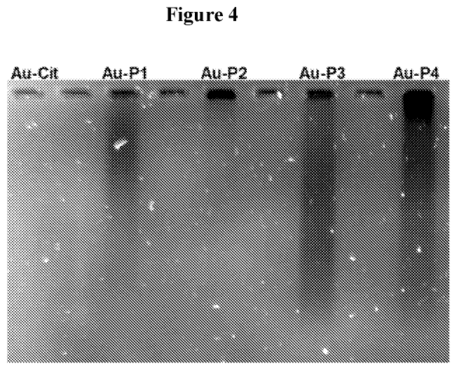

In certain embodiments, the differentially functionalized ends of the ASOs are utilized to exchange the surface capping agent of gold nanoparticles. In one embodiment, differentially functionalized thiol modified ASOs are utilized to exchange the surface capping agent of citrate stabilized gold nanoparticles. In certain embodiments, the citrate stabilized gold nanoparticles may be about 10 nm in size. In certain embodiments, the citrate stabilized gold nanoparticles may be less than 10 nm in size. In certain embodiments, the hydrodynamic diameter of the citrate-stabilized nanoparticles may be about 45 nm. In certain embodiments, the zeta potential may reveal colloidal stability of the citrate-stabilized nanoparticles with ζ=−50±6 mV. In certain embodiments, the successful surface capping of gold nanoparticles with ASOs may be confirmed from their relative change in the UV-vis absorbance spectrum, as shown in FIG. 3, and the change of the electrophoretic mobility in agarose gel electrophoresis, as shown in FIG. 4.

### Electrodes

The electrochemical biosensor disclosed herein comprises at least two electrodes. In certain embodiments, the electrochemical biosensor has at least one sensing electrode and at least one reference electrode. In other embodiments, the electrochemical biosensor has at least one sensing electrode and at least one counter electrode. The at least one sensing electrode is configured to selectively measure and detect specific biological pathogens. The sensing electrode may be a screen-printed biosensor electrode formed of metal, such as gold. In certain embodiments, the sensing electrode may be coated with, conjugated to, coupled to, bound to, incubated with or otherwise connected with the sensing element.

In some embodiments, the electrochemical biosensor comprises a reference electrode. In certain embodiments, the reference electrode may be an Ag/AgCl electrode, a hydrogen electrode, a calomel electrode, a copper-copper(II) sulfate electrode, a palladium-hydrogen electrode or a mercury-mercurous sulfate electrode.

In some embodiments, the electrochemical biosensor comprises a counter electrode. In certain embodiments, the electrochemical biosensor has a sensing electrode, a reference electrode and a counter electrode. In certain embodiments, the counter electrode, which may also be referred to as an auxiliary electrode, is an electrode which is used to close the current circuit in the electrochemical cell. In certain embodiments, the counter electrode is made of an inert material. In certain embodiments, the counter electrode does not participate in the electrochemical reaction. In certain embodiments, the total surface area of the counter electrode is higher than the total surface area of the sensing electrode. In certain embodiments, the counter electrode is comprised of platinum, gold, silver, graphite, glassy carbon or a combination thereof.

### Detecting a Target Sequence of a Biological Pathogen

Certain embodiments of methods of detecting a target gene are shown in FIG. 1. In certain embodiments, the electrochemical biosensor is part of a device that may be used for the detection of a biological pathogen. In certain embodiments, the method comprises the detection of a biological pathogen in a sample without RNA extraction or amplification. In certain embodiments, the method includes obtaining a sample from a patient. In certain embodiments, the sample may be a fluid or tissue sample. In certain embodiments, the sample may be a nasal sample, a nasopharyngeal sample or a saliva sample. In certain embodiments, the sample may be collected from a patient using a saliva swab, nasal swab, nasopharyngeal swab or other typical techniques known in the art for obtaining an RNA or DNA sample from a patient.

In certain embodiments, the method includes determining a reference measurement. In certain embodiments, determining a reference measurement includes determining at least one electrical characteristic of the electrochemical biosensor when exposed to a reference. In certain embodiments, determining a reference measurement can include using the electrochemical biosensor and an appropriate detector to measure resistance, conductivity, voltage, current, or impedance of the electrochemical biosensor when exposed to a reference. In certain embodiments, the method includes determining a reference measurement by exposing the electrochemical biosensor to a buffer, such as a lysis buffer, in a collection tube. In certain embodiments, the method includes determining a reference measurement by exposing the electrochemical biosensor to water or phosphate-buffered saline (PBS). In certain embodiments, the method includes using the reference measurement as a baseline for comparison to a sample measurement.

In certain embodiments, the method includes determining a sample measurement. In certain embodiments, determining a sample measurement includes determining at least one electrical characteristic of the electrochemical biosensor when exposed to a sample. In certain embodiments, a detector is employed to measure the at least one electrical characteristic. In certain embodiments, determining a sample measurement can include using the electrochemical biosensor to measure resistance, conductivity, voltage, current, or impedance of the electrochemical biosensor when exposed to a sample. In certain embodiments, the electrochemical biosensor is configured to measure impedance of the electrochemical biosensor when exposed to a sample. In certain embodiments, the method includes placing a sample in a collection tube before the sample measurement is determined. In certain embodiments, the collection tube contains a buffer, such as a lysis buffer. In certain embodiments, the collection tube containing a buffer, such as a lysis buffer, is the same collection tube that was used for determining a reference measurement. In certain embodiments, determining a sample measurement can include using the electrochemical biosensor to measure resistance, conductivity, voltage, current, or impedance of the electrochemical biosensor when exposed to a sample by immersing a portion of the electrochemical biosensor (e.g., the sensing electrode and reference electrode) into the buffer containing the sample.

In certain embodiments, the method includes identifying a positive sample. In certain embodiments, the method includes differentiating between a positive sample and a negative sample. In certain embodiments, identifying a positive sample includes determining the relative change of at least one measured electrical characteristic of the electrochemical biosensor when exposed to a sample from at least one measured electrical characteristic of the reference. In certain embodiments, identifying a positive sample can include comparing a change in an impedance amplitude of the sample and an impedance amplitude of the reference.

In certain embodiments, the method includes providing a signal as an output. In certain embodiments, the signal may be an amperometric signal or a cyclic voltammetry signal. In certain embodiments, the method includes providing a signal if a positive sample is identified. In certain embodiments, the electrochemical biosensing device is configured to send a signal to a recording device or external device. In certain embodiments, the electrochemical biosensing device is coupled to a detector configured to measure a signal, which in certain embodiments may be an amperometric signal or a cyclic voltammetry signal. In certain embodiments, the electrochemical biosensing device is coupled to a processing chip or microprocessor for the analysis and/or communication of results. In certain embodiments, the electrochemical biosensing device is configured to provide a signal to a potentiostat, wherein the potentiostat is used to record the signal. The potentiostat may be a portable potentiostat. In certain embodiments, the electrochemical sensing device is configured to provide a signal to an external device, such as a laptop, mobile device, smartphone, or the like, using a wired connector or wireless system (e.g., Wi-Fi, radio, Bluetooth®). Thus, the methods, electrochemical biosensors and devices disclosed herein are able to identify positive samples faster (e.g., in less than 5 minutes), cheaper, and easier (using common equipment such as potentiostats and mobile devices) compared to typical tests known in the art.

In certain embodiments, the electrochemical biosensor disclosed herein is used in a device for the detection of a biological pathogen of interest. In certain embodiments, the device includes a collection tube. In certain embodiments, the collection tube is used to house a clinical sample obtained directly from a patient. In certain embodiments, the device includes an embedded sensor, such as an embedded sensor chip. In certain embodiments, the embedded sensor is configured to analyze the sample. In certain embodiments, the device includes a communication module. In certain embodiments, the embedded sensor is configured to provide a signal to the communication module and the communication module is configured to communicate the results or data from the sensor. In still another embodiment, the communication module is a battery-operated device configured to communicate results or data from the sensor. In certain embodiments, the device is powered by a typical power source, such as a wall outlet. In certain embodiments, the communication module can include a display, such as for displaying numeric results in real-time to a user and/or for communicating results to an external device, such as a computer, laptop, mobile device (including mobile devices having an application or program for receiving results), or cloud server. In certain embodiments, the communication module can be configured to communicate results using a wired or wireless connection according to typical protocols.

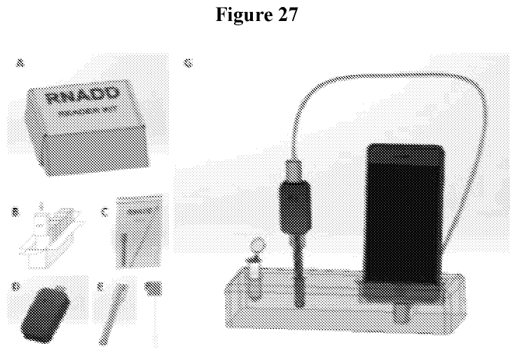

FIG. 27 shows a device having a sensor strip comprising an electrochemical biosensor in accordance with certain embodiments. In one exemplary embodiment, a method of using this device may comprise opening a package and removing a collection tube that contains lysis buffer. The method may comprise inserting the sensor strip into the collection tube and determining a reference measurement. In some embodiments, the reference measurement may be displayed on a reader display. In certain embodiments, the sensor strip is not removed from the collection tube. The method may then comprise collecting a sample using a nasal swab and inserting the nasal swab into the collection tube and mixing it into the lysis buffer. The method may then comprise waiting a predetermined amount of time (e.g., approximately 30 seconds) for the device to determine a sample measurement and display the results. The method may then include displaying a positive or negative result on the device. The method may also include displaying the viral copy number using data and/or a signal from the electrochemical biosensor. The method may then comprise removing the reader display. The method may then comprise discarding the collection tube with the swab and the sensor strip.

### Detection of Target Genes Using ASOs Bound to Nanoparticle Signal Amplifiers and a Conductor Set

In certain embodiments, the electrochemical biosensor uses ASOs bound to nanoparticle signal amplifiers and a conductor set for the detection of a biological pathogen. In certain embodiments, the nanoparticle signal amplifiers are conductive nanoparticles. In certain embodiments, the nanoparticle signal amplifiers are metal-based nanoparticles. In certain embodiments, the nanoparticle signal amplifiers are gold nanoparticles.

In certain embodiments, the electrochemical biosensor comprises a substrate. In certain embodiments, the substrate may comprise filter paper, nitrocellulose paper, ceramic and/or plastic. In certain embodiments, the substrate may comprise plastics such as polyethylene terephthalate (PET) or plexiglass. In certain embodiments, the electrochemical biosensor comprises a conductor set. In certain embodiments, the conductor set comprises at least one electrode and a conductive film, wherein the conductive film has conductive properties. In certain embodiments, the conductive film is coated on the substrate. In some embodiments, the conductive film comprises a conductive 2D material. In some embodiments, the conductive film comprises polymers, graphene nanoplatelets, graphene oxide, molybdenum disulfide, carboneous materials or carbon nanotubes. In certain embodiments, the conductive film comprises nanotubes such as single-wall nanotubes (SWNTs) or multi-wall nanotubes (MWNTs). The conductive film plays an important role in the sensing response. In some embodiments, the concentration of graphene suspension used to make the conductive film is between 5 mg/L and 20 mg/L. In some embodiments, the concentration of the graphene suspension used for making the conductive film is about 10 mg/L.

In certain embodiments, at least one electrode is deposited on the conductive film. In certain embodiments, at least one electrode is a sensing electrode. In certain embodiments, the electrode comprises a metal, such as gold. In certain embodiments, the sensing element includes ASOs connected to nanoparticle signal amplifiers. In certain embodiments, the ASOs are capped with gold nanoparticles, which are deposited on the surface of the sensing electrode. In certain embodiments, the electrochemical biosensor has a unique 2D/3D configuration, wherein the substrate, conductive film and the at least one electrode are arranged such that they are perpendicular or nearly perpendicular to the ASO capped gold nanoparticles.

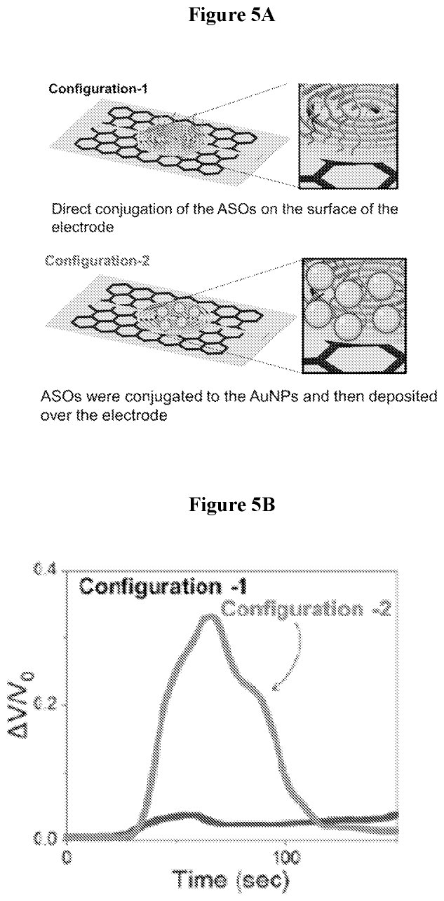

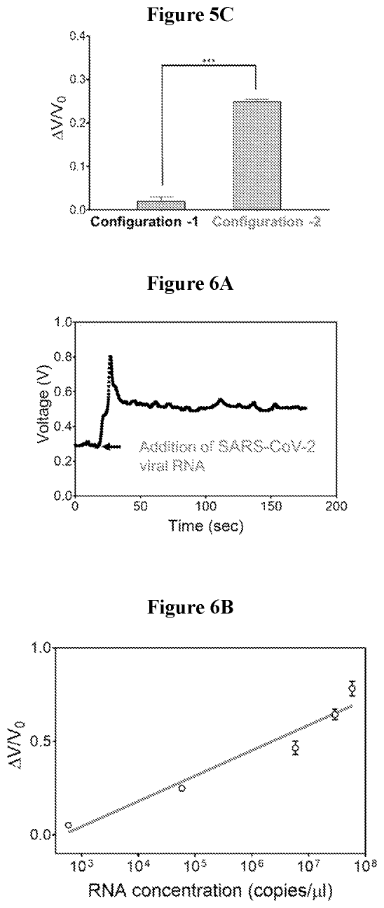

In certain embodiments, ASO capped gold nanoparticles are deposited on the surface of the sensing electrode, representing the recognition element of the sensor. In certain embodiments, using ASO capped gold nanoparticles increases the sensitivity of the electrochemical biosensor by amplifying the electrical signal due to the excellent unmatched properties of gold nanoparticles. As shown in FIGS. 5B and 5C, in certain embodiments, the sensor response to the presence of a biological pathogen is significantly higher (p<0.001) when ASO capped gold nanoparticles were used compared to the response when ASOs were directly connected with the sensing electrode. This significant increase in electrical signal was using ASO capped gold nanoparticles may also be attributed to the increased reactivity of the ASOs when capped on AuNPs and deposited over the electrode surface, favoring the electron transfer kinetics and also because the AuNPs provided a high surface area for the interaction of ASOs with the target sequence, eventually leading to signal amplification. On the other hand, the lower electrical responses observed for ASOs that were not capped with gold nanoparticles indicated decreased conductive pathways for electron transfer. Further, AuNPs, due to their conductive characteristics, contribute to these conduction pathways by accelerating the electron transfer, leading to the increased output signal in the presence of the biological pathogen.

In some embodiments, citrate-stabilized AuNPs of about 10±5 nm in size may be used to cap the ASOs. In certain embodiments, citrate-capped gold nanoparticles without any DNA barely migrate, whereas the ASO capped gold nanoparticles may migrate differentially plausibly due to their differential surface functionalization with ASOs. In some embodiments, the differential electrophoretic mobility of the gold nanoparticles indicates the differential surface charge associated with the nanoparticles.

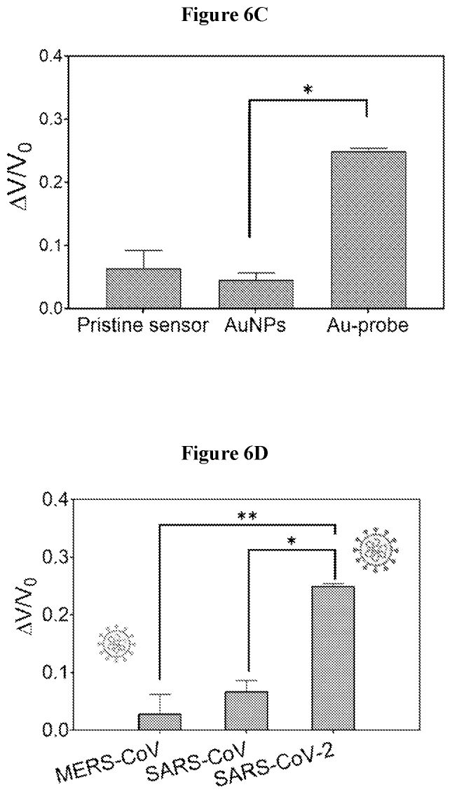

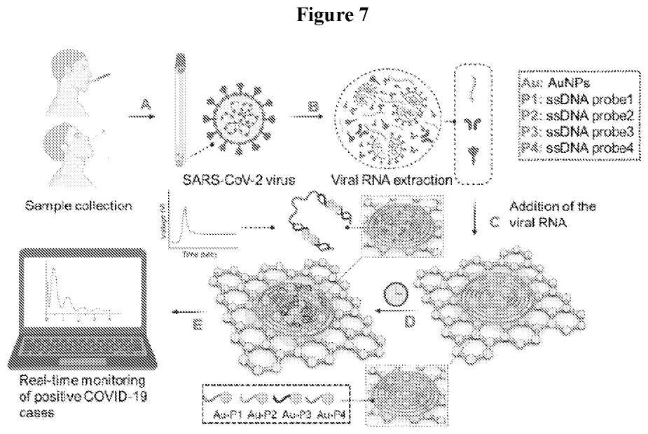

In certain embodiments, the ASO capped gold nanoparticles selectively bind with their target complementary genetic sequences and induce aggregation depending on their closely following target sequence positions. In the presence of the target genetic sequences, the specific nucleic acid molecule hybridization leads to a change in charge and electron mobility on the surface of the conductive film, which brings a change in the sensor output voltage. The change in the sensor electrical signal is observed almost instantaneously and reaches stability within a short duration of time. In some embodiments, the average response time of the electrochemical sensor is less than 5 minutes, as shown in FIG. 6A. This illustrates a representative output signal of the sensor chip as a function of time after the addition of the target gene. During hybridization, the charges at the conductive film-solution interface increase due to the hybridization of the target sequence with the ASO capped nanoparticles, which induces a variation in the conductive film potential with a positive drift.

In certain embodiments, the affinity of the ASO capped gold nanoparticles to the target sequence may be confirmed using an advanced instrumental technique such as hyperspectral spectroscopy (HSI). HSI is a label-free detection technique that can be used to confine a nanomaterial based on its hyperspectral signature by combining the power of both imaging and spectrophotometry. The HSI system is based on the enhanced dark-field microscopic (EDFM) advanced optics combined with a computational algorithm used to capture a spectrum in the range of 400 to 1000 nm at each pixel of the image. Each individual material exhibits a spectrum signature here representing the identity of the material of interest even in a blended sample. Thus, the hyperspectral can be used to create an image “map” to localize the material of interest in the tested sample.

In certain embodiments, the sensor sensitivity may be enhanced by optimizing the concentrations of the ASOs to achieve an improved electrical response. In certain embodiments, the sensor sensitivity may be enhanced by suitably designing the ASOs. In certain embodiments, the sensor sensitivity may be enhanced through the use of conductive nanoparticles, which may amplify the electrical signal. In certain embodiments, the sensitivity of the sensor may be attributed in part to the conductive film. For example, a conductive film comprised of graphene nanoplatelets has a high carrier mobility (>2000 cm² V s⁻¹), which provides enough sensitivity and adsorption capability of the charged analytes at its surface.

In certain embodiments, the electrochemical biosensor is able to detect a biological pathogen in a wide linear range from 585.4 copies/μL to 5.854×10⁷ copies/μL, with a detection limit of 6.9 copies/μL, which is significantly lower than the detection limit of other devices known in the art. In certain embodiments, the sensitivity of the electrochemical biosensor is 231 ((copies μL)⁻¹)⁻¹.

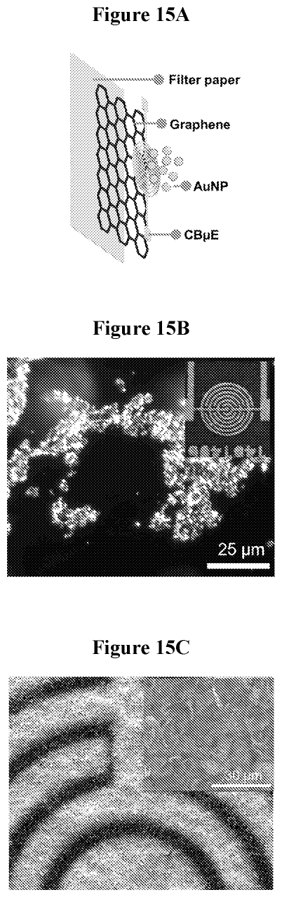

In one exemplary embodiment, a graphene suspension is first coated on filter paper to form a conductive film followed by the deposition of a gold electrode with a predefined design. A schematic of the operation of this exemplary electrochemical biosensor construct is shown in FIG. 15A. The suspension of graphene nanoplatelets provide the advantage of preventing the formation of a water-tight composite on the sensor chip, thereby giving an edge-over-edge configuration of the film at the microscopic level.

### Detection of Target Genes Using ASOs Having a Hairpin Loop Structure

In certain embodiments, the electrochemical biosensor comprises ASOs having a hairpin loop structure to detect a biological pathogen of interest. ASOs are functionalized at either the 5′ or 3′ end, such that they are capable of connecting with a sensing electrode. In certain embodiments, ASOs are functionalized with a thiol moiety or carboxylic acid at a first end (either the 5′ or 3′ end), such that the ASOs can covalently bind with the sensing electrode. In certain embodiments, in the absence of the target gene, the ASOs are in an energy-minimized state and tend to adopt a hairpin loop structure due to interactions between the base pairs of the ASOs. In the hairpin loop structure, the first and second ends of the ASOs are positioned near to the surface of the sensing electrode.

In the presence of the target gene, a complementary interaction induces a conformational change in each ASO so that the ASOs may bind to the target sequence. In certain embodiments, when an ASO binds to the target sequence, the ASO stretches to accommodate the target sequence, thus increasing the distance between the second end of the ASO and the sensing electrode. In certain embodiments, the increased distance between the second end of each ASO and the sensing electrode causes a change in electron transfer, which affects the flow of current. In certain embodiments, the change in electron transfer may be detected by cyclic voltammetry. In certain embodiments, the binding of the ASOs to the target sequence provides a signal to identify the presence

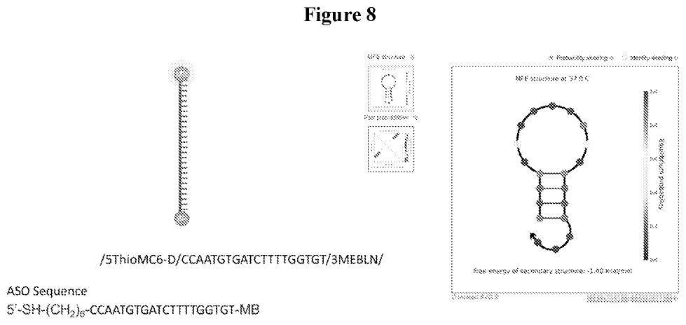

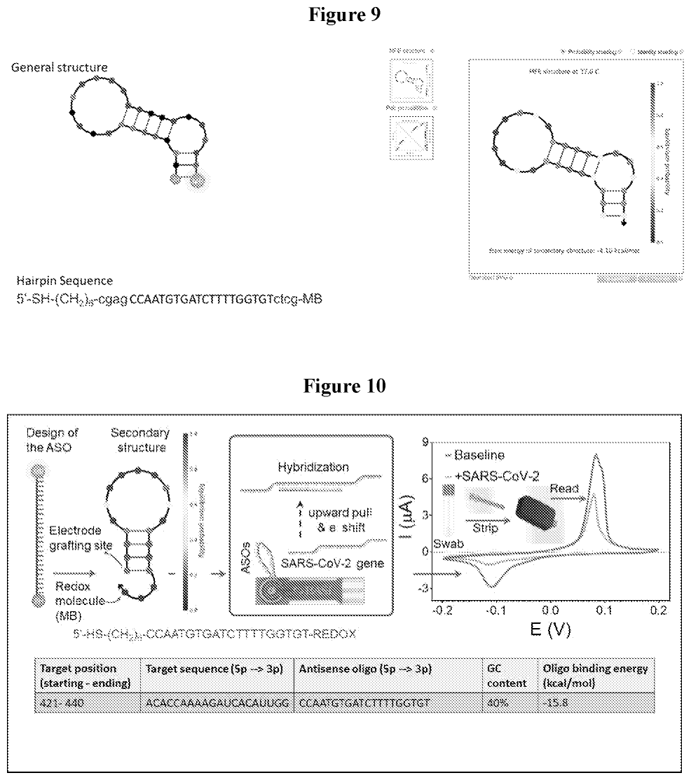

In certain embodiments, the electrochemical sensor may further comprise a redox reporter molecule. In certain embodiments, the ASO may be functionalized with a thiol group or carboxylic acid at a first end of the ASO (either the 5′ or 3′ end), and a redox reporter molecule is conjugated to the second end of the ASO. In other embodiments, the ASO may be connected to a sensing electrode at a first end of the ASO and a redox reporter molecule is conjugated to the second end of the ASO. Attention is directed to FIGS. 8 and 9, which each show an example of a thiol modified ASO that is conjugated to a redox reporter molecule (in this case, methylene blue). FIGS. 8 and 9 each provide an example of a hairpin loop structure. The ASOs shown in FIGS. 8 and 9 each have different sequences.

In the absence of the target gene, the ASO forms a hairpin loop structure, such that the redox reporter molecule is brought within proximity of the sensing electrode. In the presence of the target gene, a complementary interaction induces a conformational change in each ASO so that the ASOs may bind to the target sequence. When an ASO binds to the target sequence, the ASO unfolds to accommodate the target sequence, thus increasing the distance between the redox reporter molecule and the sensing electrode, resulting in providing a signal. In some embodiments, the binding of the ASO to the target sequence causes a change in current, as shown in FIG. 10. In some embodiments, the use of a redox reporter molecule allows electron transfer to become more pronounced, which increases the sensitivity of the electrochemical biosensor. In some embodiments, methylene blue is used as a redox reporter molecule. In certain embodiments, ferrocene is used as a redox reporter molecule. In certain embodiments, the redox reporter molecule is anthraquinone, viologen, Atto MB2 or nile blue.

### Sample Preparation

The electrochemical biosensor described herein may be used to detect a target gene in a clinical sample. In certain embodiments, a lysis step may be performed on the sample. Examples of lysis methods know in the art include thermal lysis, alkaline lysis, detergent lysis, and enzymatic cell lysis. In an embodiment, detergent lysis is employed, and a detergent is added to the clinical sample. Suitable detergents are known in the art, including: sodium dodecyl sulphate (SDS), Triton™ X 100, Triton™ X 114, NP-40, Tween™ 20, Tween™ 80, cetyltrimethylammonium bromide (CTAB), CHAPS and CHAPSO. In one embodiment, nucleic acids are extracted from a clinical sample prior to detection of the target gene. Methods for extraction of nucleic acids are known in the art. For example, if the nucleic acid of interest is RNA, suitable methods of RNA extraction include hot acid phenol, Qiamp DSP Virus Spin kit, Total RNA Purification Kit, RNEasy™ Mini Kit (Qiagen™), Illustra™ RNAspin Mini RNA Isolation Kit (GE™), Viral Nucleic Acid (DNA/RNA) Extraction Kit I, and EXTRAzol/TRIzoI™. Alternatively, no extraction step may be performed prior to detection of the target gene.

### Device or Test Kit

The electrochemical biosensor described herein may be provided as part of a device or a test kit for the detection of one or more biological pathogens of interest.

Such devices and kits may include a sensing element comprising suitable ASOs designed and chosen specifically to bind a nucleic acid sequence in a target gene. In certain embodiments, the ASOs are designed and chosen in pairs to bind closely spaced nucleic acid sequences in a target gene. Such kits may also include at least two electrodes. Such kits may also include a detector for measuring at least one electrical characteristic from at least one electrode. Such kits may optionally include a communication module or processor.

Such devices and kits may optionally include additional components, including a sample collection apparatus such as a swab or collection vial, a sample collection and/or storage and/or preservation buffer or solution, lysis buffer or detergent, components and reagents for nucleic acid extraction, and/or a detection apparatus. Examples of such optional additional components are known in the art. Examples of suitable sample collection and/or storage and/or preservation buffers or solutions include viral transport medium and PrimeStore™ MTM. Examples of suitable lysis detergents include sodium dodecyl sulphate (SDS), Triton X 100, Triton X 114, NP-40, Tween 20, Tween 80, cetyltrimethylammonium bromide (CTAB), CHAPS and CHAPSO. If the nucleic acid of interest is RNA, suitable methods of RNA extraction include hot acid phenol, Qiamp DSP Virus Spin kit, Total RNA Purification Kit RNEasy Mini Kit (Qiagen), Illustra RNAspin Mini RNA Isolation Kit (GE), Viral Nucleic Acid (DNA/RNA) Extraction Kit I, and EXTRAzol/TRIzol.

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques, electrochemistry, physics, and pharmacology, within the skill of the art. Such techniques are explained fully in the literature.

### Example 1. Design and Selection of Antisense Oligonucleotides (ASOs)

During the current spread of COVID-19 causative virus, SARS-CoV-2, scientists have discovered three regions among the SARS related viral genomes which had conserved sequences. These sequences are: (a) RdRP gene (RNA-dependent RNA polymerase gene) responsible for the open reading frame ORF1ab region, (b) E gene (envelope protein gene), and (c) N gene (nucleocapsid phosphoprotein gene). The analytical sensitivity of both the RdRP and E genes was demonstrated to be quite high (technical limit of detection of 3.6 and 3.9 copies per reaction), while the sensitivity for N gene observed to be weaker (8.3 copies per reaction). This leaves an enormous area for the improvement of biosensors targeted for N gene sequence of SARS-CoV-2. The analytical sensitivity of the biosensor can be improved by simultaneous targeting of multiple genetic regions within the same gene sequence, which will add to features of the biosensor. This will also increase the feasibility of the assay even if one region of the viral gene undergoes mutation during its current spread. Therefore, the N gene sequence of SARS-CoV-2 was targeted.

The N gene (nucleocapsid phosphoprotein gene) was undertaken herein as the target gene sequence for the selective detection of SARS-CoV-2 isolate 2019-nCoV/USA-WA1-A12/2020 and a set of ASOs were predicted using the following methodology.

The target N-gene sequence of SARS-CoV-2 SEQ ID NO 1 was supplied to a software for statistical folding of nucleic acids and studies of regulatory RNAs, Soligo, and the ASOs were predicted maintaining the folding temperature as 37° C. and ionic conditions of 1 M sodium chloride for a preferred length of ASO as 20 nucleotide bases. The filter criteria were set as follows: [1] 40%≤GC %≤60%; [2] Elimination of target sequences with GGGG; [3] Average unpaired probability of the ASOs for target site nucleotides to be ≥0.5; [4] Considering the threshold probability of above 0.5, all sites targeted to the peak in the accessibility profile are ranked by their average unpaired probability (the higher the better); [5] Among sites satisfying criteria 1-4, the top 20 ones with the highest average unpaired probability will be considered. The average unpaired probability was also used in filter criteria 3, 4 and 5 to reduce the number of reported sites in order to optimize the disruption energy calculation in the web servers. [6] Further, the binding energy of the ASOs were also compared with the target sequence where the binding energy cutoff for the selection of ASOs was kept at ≤−8 kcal/mol.

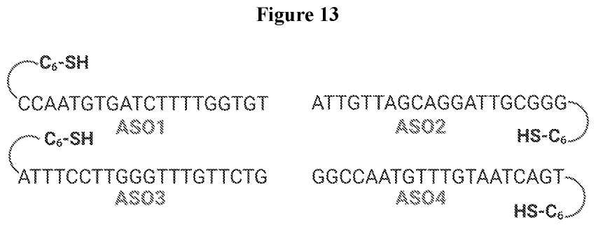

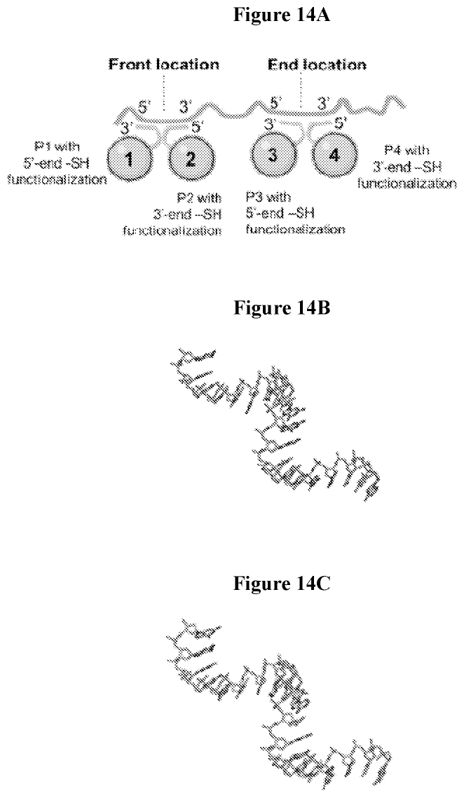

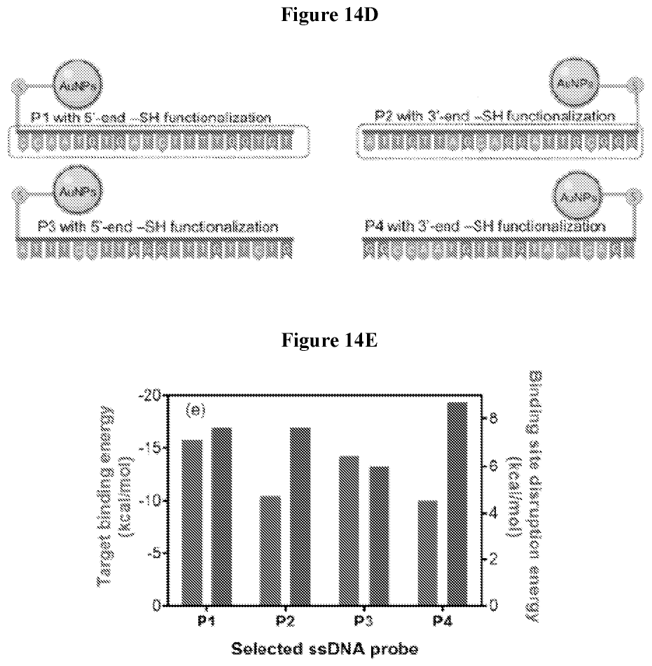

Among the predicted ASO sequences, four of the ASOs were selected based on their comparative binding disruption energies and binding energies with the target sequence (as shown in Table 1, FIG. 13 and FIG. 14E). One of the other parameters behind the selection of these four ASO sequences was their closely following target position. The ASOs were then differentially functionalized as shown in FIG. 14A: P2 and P4 were functionalized with thiol moiety at the 5′ end, whereas ASO2 and ASO4 were functionalized with thiol moiety at the 3′ end.

TABLE 1
Selected ASO sequences targeted for the N-gene of SARS-COV-2.

| | | | | | Binding | |
|---|---|---|---|---|---|---|
| Starting | Ending | | | | site | |
| target | target | Target | Antisense | | disruption | Binding |
| posi- | posi- | sequence | oligo | GC | energy | energy |
| tion | tion | (5p → 3p) | (5p → 3p) | content | (kcal/mol) | (kcal/mol) |
| 421 | 440 | ACACCAAAAG | CCAATGTGATC | 40.0% | 7.6 | −15.8 |
| | | AUCACAUUGG | TTTTGGTGT | | | |
| | | (SEQ ID NO 2) | (P1) | | | |
| | | | (SEQ ID NO 6) | | | |
| 443 | 462 | CCCGCAAUCC | ATTGTTAGCAG | 50.0% | 7.6 | −10.4 |
| | | UGCUAACAAU | GATTGCGGG | | | |
| | | (SEQ ID NO 3) | (P2) | | | |
| | | | (SEQ ID NO 7) | | | |
| 836 | 855 | CAGAACAAAC | ATTTCCTTGGG | 40.0% | 6.0 | −14.3 |
| | | CCAAGGAAAU | TTTGTTCTG | | | |
| | | (SEQ ID NO 4) | (P3) | | | |
| | | | (SEQ ID NO 8) | | | |
| 886 | 905 | ACUGAUUACA | GGCCAATGTTT | 40.0% | 8.7 | −10.0 |
| | | AACAUUGGCC | GTAATCAGT | | | |
| | | (SEQ ID NO 5) | (P4) | | | |
| | | | (SEQ ID NO 9) | | | |

### Example 2. Synthesis of Citrate-Stabilized Gold Nanoparticles

A solution of 2.2 mM of sodium citrate was taken in ultrapure water (150 mL) and boiled to reflux for 15 minutes with continuous stirring. A solution of 1 mL of chloroauric acid (25 mM) was then added to the refluxing solution. The solution was stirred for a duration of 20 minutes. The resulting citrate-stabilized gold nanoparticles (AuNPs) were well suspended in water.

### Example 3. Formation of ASO-Capped Gold Nanoparticles

The citrate-stabilized AuNPs with a concentration of about 3×10¹⁰ particle/mL, as measured through ZetaView software, were treated with ASOs at three different concentrations, i.e., 0.5, 1, and 2 μM from a stock of 200 μM for each of the four ASOs. The mixture was then kept at room temperature for 30 minutes with constant stirring. Any excess uncapped ASO was then removed from the solution by centrifugation at 20 000 rcf for 15 minutes. The residue was finally resuspended in an equivalent volume of water. Accordingly, twelve different samples for four of the ASOs at three different concentrations were prepared and defined as Au-P_{xL}, Au-P_{xM} and Au-P_{xH}, where x defines the number of ASOs as 1, 2, 3 or 4 and L, M and H are representative of low (0.5 μM), medium (1 μM) and high (2 μM) concentrations of ASOs, respectively. The nanoparticles were kept at 4° C. for future use.

The successful surface capping of AuNPs with ssDNA probes was confirmed from their relative change in the UV-vis absorbance spectrum as shown in FIG. 3, the change of the electrophoretic mobility in agarose gel electrophoresis as shown in FIG. 4. Citrate-capped AuNPs without any DNA barely migrated, whereas the ssDNA-capped AuNPs migrated differentially plausibly due to their differential surface functionalization with ssDNA probes. The differential electrophoretic mobility of the gold nanoparticles indicated the differential surface charge associated with the nanoparticles. It can therefore be inferred that more ssDNA functionalization will lead to a more surface negative charge and hence more electrophoretic mobility. Thus, it can be said that the ssDNA functionalization is more for Au-P_{3H}, followed by Au-P_{4M}, Au-P_{1M}, and Au-P_{2L}.

### Example 5. Cell Culture, Isolation of RNA

Cercopithecus aethiops kidney epithelial cells (Vero E6) were cultured in Eagle's minimum essential medium at standard conditions with the supplementation of 10% fetal bovine serum at 37° C. The culture was maintained by trypsinization of the cells with 0.25% (w/v) trypsin-0.53 mM EDTA solution.

Severe acute respiratory syndrome-related coronavirus (SARS-CoV-2), isolate USA-WA1/2020, NR-52287, was obtained from BEI Resources, NIAD, NIH. The sample consists of a crude preparation of gamma-irradiated cell lysate and supernatant from Vero cells infected with SARS-CoV-2 isolate. Separately, NR-50549 consisting of a gamma-irradiated cell lysate and supernatant from Vero cells infected with MERS-CoV, EMC/2012, was obtained from BEI Resources, NIAID, NIH. The Vero cells before and after the viral infection were lysed by the addition of TRlzol reagent (1 mL) and aspirated. The total RNA was then extracted and purified from the cell lysate, and the RNA concentration was measured by a Nanodrop. The purified SARSCoV-2 and MERS-CoV viral RNA concentrations were found to be 35.9 and 4 ng/μL, respectively, with the RNA concentration of noninfected Vero cells being 92.6 ng/μL.

### Example 6: Fabrication of the Electrochemical Biosensor

Graphene nanoplatelets (GNPs) and molecular biology grade water were purchased from STREM Chemicals, USA, and Sigma-Aldrich, respectively. Briefly, GNPs with a width of about 6-8 nm and length of about 25 μm (100 mg) were added to 10 mL of molecular biology grade water to prepare a suspension. The resulting suspension was probe sonicated at 37° C. at an amplitude of 4, with a pulse rate of 5 seconds on and 1 second off for 8 cycles (about 4 hours) with intermittent cycles of 30 minutes. To increase the uniformity of resulting mixture over filter paper (Whatman filter paper about 90 mm) with minimum surface defects, a spin-coating technique (Laurell Technologies, North Wales, PA, USA) was utilized at 150 rpm for 45 seconds, 300 rpm for 600 seconds, and 600 rpm for 120 seconds. The spin-coated filter papers were then dried for 10 minutes under continuous nitrogen flow and dried overnight under a vacuum desiccator at room temperature. Finally, an electron beam evaporator (Temescal Systems FC-2000, Livermore, CA, USA) was used to deposit the micro-Au electrodes with a thickness of 50 nm using a shadow mask with a predefined pattern. The shadow mask design was initially created in AutoCAD 2014 and printed at the facility of Photo-Sciences Inc., Torrance, CA, with a thickness of 0.025 mm on a nickel-brass. FIG. 15A shows an image illustrating the layer-by-layer structure of the electrochemical biosensor.

### Example 7. Protocol for Sample Standardization

The as-synthesized ASO stabilized AuNPs were first sonicated in a bath sonicator for 5 minutes at room temperature before each use. The solutions were then vortexed for 2 minutes, and the individual ASO-capped AuNPs, 8 μL from each solution, were added on top of the gold electrode. The sensor chip was left at room temperature for 30 minutes with no sign of moisture. On the other hand, for the preparation of Pₘᵢₓ, equivalent portions from each of the probes (P_{1M}, P_{2L}, P_{3H} and P_{4M}) were taken, mixed, and vortexed properly; that is, for a solution of 8 μL of Pₘᵢₓ, 2 μL from each of the selected ASO formulations was taken. The RNA samples (2 μL) with different concentrations were then added on top of the electrodes.

### Example 8. Electrochemical Data, Absorbance Spectra, Measurement of Hydrodynamic Diameter, Raman Spectroscopy, Hyperspectral Microscopy

The current-voltage electrochemical data were collected using a homemade circuit consisting of a signal conditioner circuit and a microcontroller. Each of the individual experiments was repeated at least six times, and an average value of the same has been reported. The obtained signals have been used to evaluate sensor performance. The limit of detection has been calculated based on the following equation: LOD=3.3S_{XY}/slope.

The absorbance spectra were recorded on a VWR UV-vis spectrophotometer.

The hydrodynamic diameters of the particles were monitored on a particle tracking analyzer (ZetaView Particle Metrix). The hydrodynamic diameters of ASO-capped AuNPs before and after the addition of RNA 5.854×10⁴ were measured in triplicate.

For Raman spectroscopy, the paper-based electrochemical sensor was placed on a microscope glass slide and imaged using a fiber-optic probe with an integrated 532 nm Raman filter set; the probe was connected to a portable Raman spectrometer (Yvon Jobin LabRam ARAMIS).

For hyperspectral microscopy, a film of graphene, as described in Example 6, was deposited on a glass slide. Next, a mixture of the gold nanoparticles capped with the four ASOs (Pₘᵢₓ) was deposited on the graphene film to mimic the sensor chip. The dark-field imaging and hyperspectral data were recorded with dark-field optical microscope (CytoViva) system of the graphene film alone, with the ASO-capped gold nanoparticles in the absence of the COVID-19 viral RNA and after the addition of 1 ng/mL of SARS-CoV-2 RNA.

### Example 9. Cross-Reactivity Experiment

To perform a cross-reactivity experiment, we obtained formaldehyde and UV-inactivated, purified, SARS coronavirus (NR-3883) from BEI Resources and isolated the viral RNA using the manufacturer's protocol as detailed in the Zymo Research Quick-RNA viral kit (R1035). The extracted RNA was tested using the developed sensor chip of Examples 1-7.

### Example 10. Experimental Setup and Real-Time Monitoring

The fabricated sensors prepared according to Examples 1-7 were interfaced with an in-house-built benchtop electrical circuit. The electrical circuit is composed of an Arduino UNO. The microcontroller (ATmega328P) was reconfigured to control the input voltage of 5 V, and a laptop was used to record the data in real time.

### Example 11. Preparation of Clinical Samples

The clinical samples tested in this work were collected as part of the registered protocols approved by the Institutional Review Board (IRB) of the University of Maryland, Baltimore. Samples of nasopharyngeal swabs were stored in viral transfer media. The clinical samples were heat-inactivated by heating the samples at 65° C. for 30 min, and then the samples were stored at −80° C. for future use.

### Example 12. Procedure for the LabGun RT-PCR Assay

The LabGun COVID-19 RT-PCR kit is optimized to detect RNA from SARS-CoV-2 in nasopharyngeal swabs collected from patients with signs and symptoms of COVID-19. Briefly, nucleic acids were first isolated and purified by the QIAamp viral RNA mini extraction kit. The purified genetic material was then amplified using the LabGun COVID-19 RT-PCR kit. During the PCR extension phase, the probe was degraded by the 5′ nuclease activity of Taq polymerase, resulting in the reporter dye being differentiated from the quencher dye. This induces an increase in fluorescence emission. At each cycle, more reporter dye molecules are cleaved from their probes and increase fluorescence. The increase in fluorescence intensity is generally monitored at each PCR cycle by the RT-PCR instrument. It is expected that the samples without the presence of SARS-CoV-2 would not provide any significant increase in emission, while only the COVID-19 positive samples reflect an increment in fluorescence intensity.

### Example 13. Characterization of the Paper-Based Electrochemical Biosensor

FIG. 15B depicts the dark-field image of the graphene film prepared in accordance with Examples 1-7 with AuNPs in the absence of a biological pathogen, as well as a photograph of the developed chip. FIG. 15C shows the scanning electron microscopic (SEM) image of the sensor chip of the exemplary embodiment, which shows both the edge-over-edge microscopic configuration of the graphene nanoplatelets and the pattern of gold electrodes.

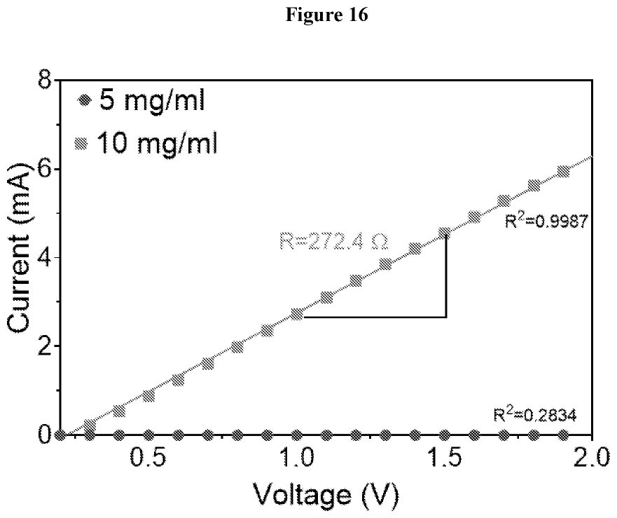

To optimize the graphene film, the concentration of the graphene suspension used for making the graphene film has been varied and the resistance of the graphene film on the filter paper was measured directly. Three different concentrations of graphene platelets (i.e., 5, 10, and 20 mg/mL) have been used to make three graphene films using the same procedure as described in Example 6. A thick and viscous suspension was formed while utilizing a graphene concentration of 20 mg/mL, which makes it difficult to form a uniformly coated surface. On the other hand, the texture of the suspension having a concentration of 10 or 5 mg/mL was reasonable to uniformly coat the filter paper surface. FIG. 16 illustrates the V-I response of the graphene film made from two different graphene concentrations. The 5 mg/mL film showed a nonlinear V-I response (R²=0.28) (i.e., not obeying Ohm's law) and very low response when compared to the film made from the 10 mg/mL suspension. Moreover, the 10 mg/mL film shows a linear V-I response (R²=0.99) and with a base resistance of 272.4 ohms. Therefore, the graphene film from a 10 mg/mL suspension was used to make the sensor chip.

### Example 14. Characterization of the ASO-Capped Gold Nanoparticles (AuNPs) for the Sensitive Detection of SARS-CoV-2

The differentially functionalized thiol-modified ASOs from Example 1 were utilized to exchange the surface capping agent of the citrate stabilized gold nanoparticles. The anhydrate particle diameter of the as-synthesized gold nanoparticles was determined using transmission electron microscopy. The hydrodynamic diameter of the citrate-stabilized nanoparticles was found to be 44.7±5.9 nm, and the zeta potential measurement revealed the colloidal stability of these particles with ζ=−50.24±6.07 mV.

To achieve selectivity and sensitivity during the recognition of SARS-CoV-2 genetic material, ASO capped AuNPs were deposited on the surface of the sensor platform in accordance with Examples 1-7. Raman spectra have been collected to further characterize the sensor chip. As shown in FIGS. 1f and g, the plain sensor shows a high I_{G}/I_{D} ratio in the presence of the gold electrode compared to the absence of it, with a higher 2D peak in the graphene region, confirming the successful mounting of the gold electrodes on top of the graphene film. Further, as shown in FIGS. 1e and h, it can be seen from the Raman spectral analysis that the graphene base provides a graphitic (sp²) to diamond (sp³) intensity ratio (I_{G}/I_{D}) of 1.74, while the sensor immobilized with the ASO capped gold nanoparticle has an I_{G}/I_{D} value of 1.56. This decrease in I_{G}/I_{D} value can be attributed to the surface-enhanced Raman scattering (SERS) caused by the presence of the AuNPs on gold electrodes. Furthermore, the graphitic wavenumber is also downshifted from 1584 to 1581 cm⁻¹, which indicates the increased π-π interactions between the oligonucleotide (ssDNA) and graphene. Once the viral sample encounters the sensor, viral RNA hybridizes with the AuNP capped ASOs, allowing a more accurate electrochemical measurement.

### Example 15. Signal Amplification Using Gold Nanoparticles Capped with ASOs

The designed antisense oligonucleotides can be incorporated on the sensor chip in two different configurations, as shown in FIG. 5A. The antisense probes can be conjugated directly to the gold electrode surface (configuration 1) or can be utilized to cap the surface of gold nanoparticles, which will then be deposited at the surface of the sensor platform (configuration 2). FIG. 5B shows the sensors response after the addition of 5.854×10⁷ of SARS-CoV-2 viral RNA. Configuration 1, in which ASOs were thiolated on the gold electrode, provides a low response signal compared to the configuration 2. As shown in FIG. 5C, the sensor response to the SARS-CoV-2 RNA in configuration 2 was found to be significantly higher (p<0.001) compared to the response in configuration 1. This significant increase in electrical signal in the presence of AuNPs (i.e., configuration 2) may also be attributed to the increased reactivity of the ASOs when capped on AuNPs and deposited over the electrode surface, favoring the electron transfer kinetics and also because the AuNPs provided a high surface area for the interaction of ssDNA probes with the viral RNA, eventually leading to signal amplification. On the other hand, the lower electrical responses observed for configuration 1 indicated decreased conductive pathways for electron transfer. Further, AuNPs, due to their excellent conductive characteristics, contribute to these conduction pathways by accelerating the electron transfer, leading to the increased output signal in the presence of SARS-CoV-2 viral RNA. Therefore, the sensor with configuration 2, consisting of ASO-capped AuNPs deposited over the gold electrode, has been used for all our future electrochemical studies.

### Example 16. Optimization of the ASO/AuNPs Ratio

The sensitivity of the sensor is highly dependent on the recognition element (i.e., the ASOs). The density of the ASOs immobilized on the sensor surface is an essential factor affecting the sensitivity and is anticipated to play a role in the sensor response. The developed platform can thus be represented by a resistive sensor model in which the change in the voltage across the sensor chip is given by

$$Δ V = R \frac{dq}{dt} \qquad ( 1 )$$

where ΔV is the change in the voltage across the sensor chip, R is the resistance, and q is the charge.

We have used the Langmuir model to fit the kinetics of binding as in the surface plasmon resonance (SPR) experiments in which this model has been widely explored at high flow speeds. The dissociation to the formation of the binding complex RNA-DNA with an immobilized ASO (D) at the end of the injunction can be given by

$$\frac{{d [ RD ]}_{t}}{t} = {{k}_{d} [ RD ]}_{t} \qquad ( 2 )$$

where [RD]ₜ is the surface density of the binding complex between the viral RNA and ASOs, [R] is the concentration of the target RNA, and k_{d} is the dissociation rate constant. Equation 2 is a first-order equation that can be solved analytically to provide eq 3:

$${[ RD ]}_{t} = \frac{{{k}_{a} [ D ]}_{max} [ R ]}{{k}_{a} [ R ] + {k}_{d}} {e}^{- {k}_{d} t} \qquad ( 3 )$$

where kₐ is the association rate constant and [D]ₘₐₓ is the maximum number of ASOs available for binding per surface area.

The change in the surface charges (Δq) can be represented by Q_{RNA}S[RD]ₜ, where Q_{RNA} is the electric charge caused by the adsorbed RNA unit to the sensor chip per surface density, and S is the graphene surface area. Then, eq 1 can be written as:

$$\begin{matrix}Δ V = R \frac{dq}{dt} = {Q}_{RNA} S \frac{{d [ RD ]}_{t}}{dt} & ( 4 ) \\ Δ V = {RQ}_{RNA} {Sk}_{d} \frac{{{k}_{a} [ D ]}_{max} [ R ]}{{k}_{a} [ R ] + {k}_{d}} {e}^{- {k}_{d} t} & ( 5 )\end{matrix}$$

Thus, from eq 5, it may be presumed that maximum response will be obtained from an immobilized density of the maximum number of ASOs, [D]ₘₐₓ. Based on eq 5, a higher response level is thus anticipated when more ASOs are immobilized on the surface. However, the hybridization kinetics of the ASOs with their complementary target can be significantly altered when high densities of ASOs are immobilized. High ASO density may lead to slower response time or nonlinear sensor response due to the unnecessarily complicated binding behavior, which rises at high probe density. Several experiments have, therefore, been conducted using the electrochemical chip to find the optimum concentration of the ASOs. Three different concentrations of each of the four ASOs prepared in accordance with Example 1 (i.e., P₁, P₂, P₃, and P₄) were used to cap the AuNPs, and the corresponding electrical performances of the sensors were investigated with a fixed concentration of the viral SARS-CoV-2 RNA load. The three different concentrations were designated as high (H), moderate (M), and low (L) level, which is equivalent to 0.5, 1, and 2 μM of the ssDNA, respectively, in accordance with Example 3.

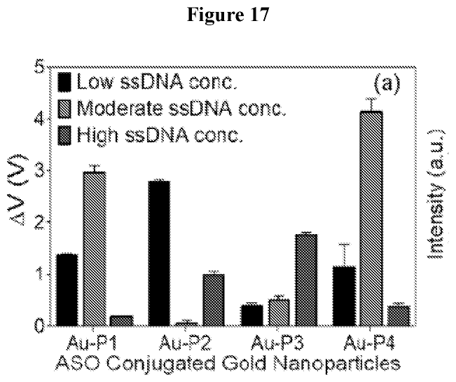

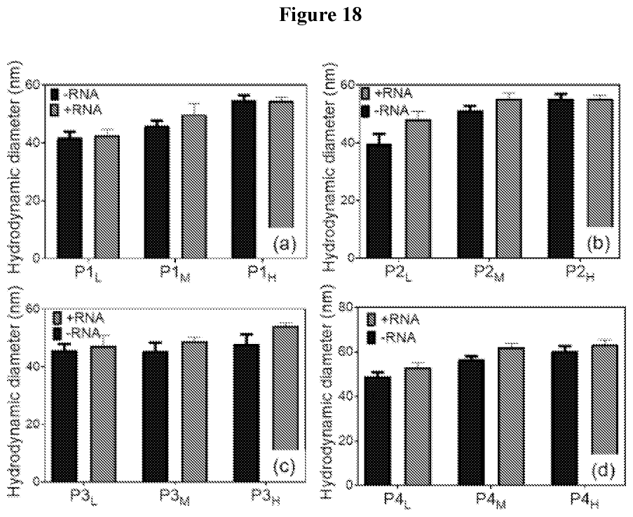

FIG. 17 illustrates the electrochemical sensor response for each of the AuNPs functionalized with ASOs toward the viral RNA at three different ASO/AuNPs ratio. The moderate ratio of P₁ (Au-P_{1M}) exhibits the highest change in the signal output, whereas the lowest ratio of P₂ (Au-P_{2L}) was found to be the optimum concentration for this probe, which shows the highest change in the sensor output compared to other concentrations. On the other hand, the highest ratio of P₃ (Au-P_{3H}) was found to be the optimum, and finally, the moderate ratio of P₄ (Au-P_{4M}) provided the best performance. The nanoparticle tracking analysis using ZetaView followed a similar trend in which the increase in hydrodynamic diameter after the addition of SARS-CoV-2 viral RNA load was found to be the highest for moderate P₁ (Au-P_{1M}), low P₂ (Au-P_{2L}), high P₃ (Au-P_{3H}), and moderate P₄ (Au-P_{4M}), as shown in FIG. 18.

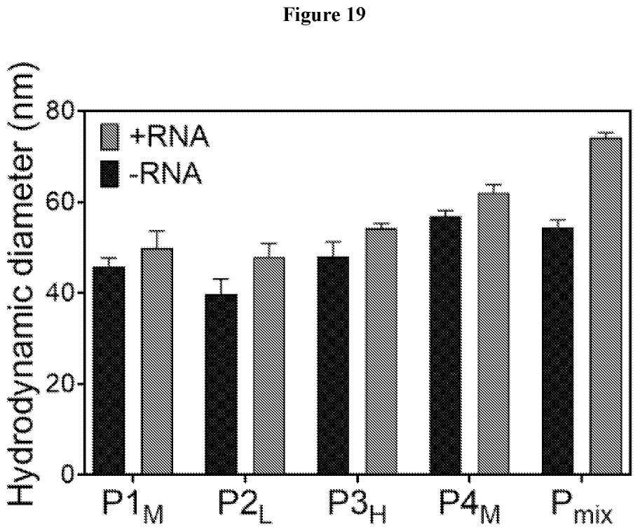

These constructs, Au-P_{1M}, Au-P_{2L}, Au-P_{3H}, and Au-P_{4M}, were then considered to build a sensitive platform for the selective recognition of SARS-CoV-2 viral RNA. The average number of ASOs conjugated to each AuNP has been quantified and found to be 6.05, 5.05, 29.8, and 20.12 for Au-P_{1M}, Au-P_{2L}, Au-P_{3H}, and Au-P_{4M}, respectively. Accordingly, as shown in FIG. 17, all the four probes (i.e., Au-P_{1M}, Au-P_{2L}, Au-P_{3H}, and Au-P_{4M}) were mixed (Au-Pₘᵢₓ) in an equivalent amount with each other to further improve the DNA-RNA hybridization propensity, as shown in FIG. 19, and enhance the analytical sensitivity of the electrochemical sensor toward the selective detection of SARSCoV-2 viral RNA.

### Example 17. Optimization of the Size of the Gold Nanoparticles

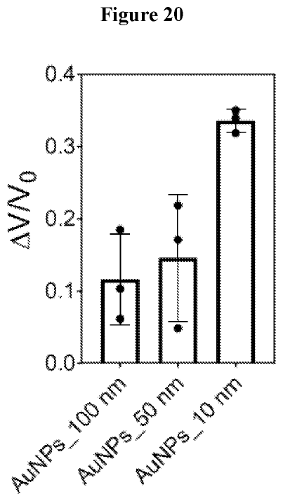

To evaluate the effect of the gold nanoparticles' size on the sensor performance, two additional gold nanoparticles with sizes of 50±5 nm and 100±10 nm have been tested in addition to the original size (10±5 nm). Each gold nanoparticle has been capped with one of the four ssDNA probes based on the previous optimum value (i.e., Au-P_{1M}, Au-P_{2L}, Au-P_{3H}, and Au-P_{4M}). An equal amount of each Au_{Xnm}-Px was mixed to make Au_{Xnm}-Pₘᵢₓ and used to make the sensor chip by depositing the mixture on the sensor surface. The response toward 10⁶ copies/μL of SARS-CoV-2 viral RNA has been recorded from the sensor chip with Au₁₀ₙₘ-Pₘᵢₓ, Au₅₀ₙₘ-Pₘᵢₓ, and Au_{100 nm}-Pₘᵢₓ. FIG. 20 shows that the 10 nm AuNPs exhibit the optimum response compared to the other nanoparticles, 50 and 100 nm in size. It was apparent that larger nanoparticles at sub-nanometer gaps have lower DNA density, in which the DNA density is higher for the small particles and decreases as the nanoparticle size increases. Accordingly, we have received a better response from the smaller sized gold nanoparticles than the larger ones.

### Example 18. Signal Amplification Using Gold Nanoparticles

To further confirm the exceptional affinity of the ssDNA probes to the target segment in the viral RNA, an advanced instrumental technique such as hyperspectral spectroscopy was used. Hyperspectral imaging (HSI) is a label-free detection technique that can be used to confine a nanomaterial based on its hyperspectral signature by combining the power of both imaging and spectrophotometry. The HSI system is based on the enhanced dark-field microscopic (EDFM) advanced optics combined with a computational algorithm used to capture a spectrum in the range of 400 to 1000 nm at each pixel of the image. Each individual material exhibits a spectrum signature here representing the identity of the material of interest even in a blended sample. Thus, the hyperspectral can be used to create an image “map” to localize the material of interest in the tested sample. Therefore, EDFM-HSI mapping has widely been confirmed to accurately localize and identify the nanomaterials in samples more efficiently compared to the other conventional approaches. To this end, the electrochemical chip, as described in Examples 1-7, was prepared on a glass slide and investigated under a hyperspectral imaging microscope to reveal the selectivity and efficient hybridization of the ssDNA probes with the viral RNA at a microscopic level. A red-shift of ˜150 nm was observed in the hyperspectral image following the addition of the SARS-CoV-2 RNA to the graphene-Au-Pₘᵢₓ-based platform. The shift in the spectral peaks after the addition of the target RNA confirmed the successful hybridization of ssDNA probes with its complementary region in N-gene sequence within the electrochemical sensor chip.

To gain more insight about the localization and distribution of each ASO-capped gold nanoparticles when bound to the viral RNA, the hyperspectral image mapping was explored. In the absence of the SARS-CoV-2 RNA, the Au probes are found to be randomly distributed within the graphene film. However, in the presence of the viral RNA, the Au probes are arranged where Au-P1 and Au-P2 were found close to each other, and Au-P3 and Au-P4 were observed to be in proximity, which supports the specific binding of the ssDNA probe to its target. Further, it can be proposed that Au-P1 was found to be the most abundant, followed by Au-P4, Au-P3, and Au-P2 among the ssDNA probe-capped AuNPs while considering the zone where Au probes are accumulated in the presence of SARSCoV-2 viral RNA. This can be explained from the comparative target RNA sequence binding energies and binding disruption energies of the four different antisense oligonucleotides, as shown in FIG. 14E.

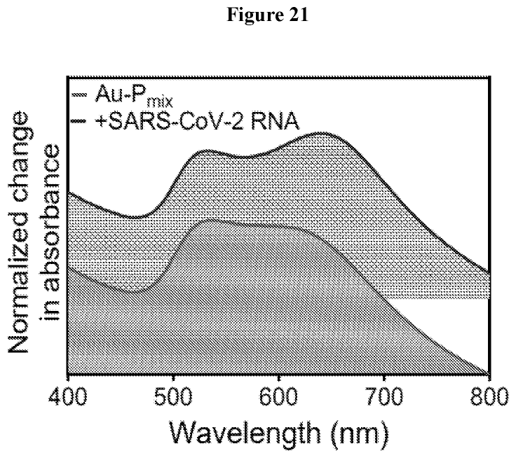

The UV-vis spectrum confirmed the sensitive and selective binding of the ssDNA-capped AuNPs to the SARS-CoV-2 viral RNA, as shown in FIG. 21.

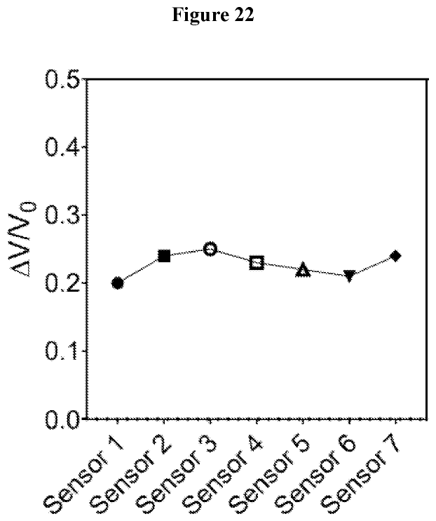

To evaluate the effect of the gold nanoparticles' distribution on the sensor's analytical performance, we assessed the variation in the sensor response to the same viral load using different sensors; the gold nanoparticles were randomly distributed on each of them. Gold nanoparticles were deposited on the surface of multiple sensor chips from which the deposition process was independent for each of them. The response of seven sensor chips was recorded against an equal concentration of SARS-CoV-2 RNA (˜1×10⁵ copies/μL). No significant difference was observed between the different sensors. The average response of the seven sensors was found to be 0.23, with a standard deviation of 0.02. These results indicated that the variation in the AuNP distribution due to the deposition method had no obvious effect on the sensor performance, as shown in FIG. 22.

### Example 19. Real-Time Detection of SARS-CoV-2 Viral RNA

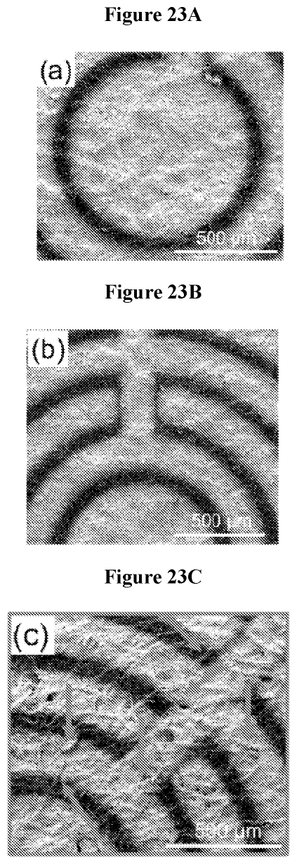

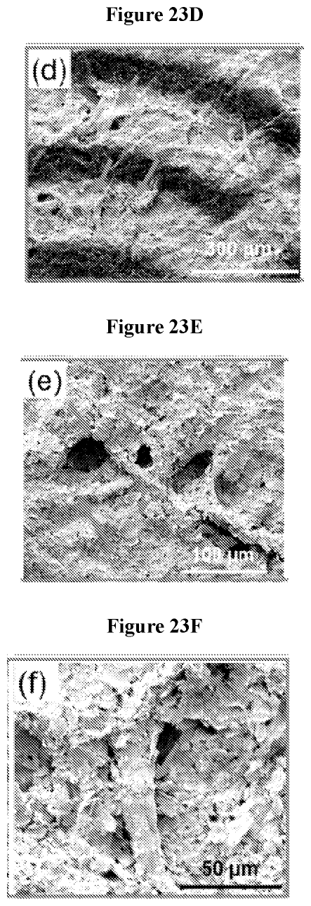

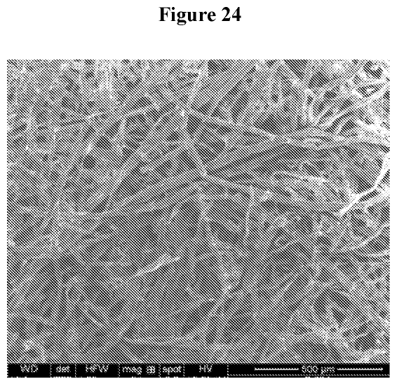

The real-time recording of electrical signals allowed the sensitive detection of SARS-CoV-2 genetic material in the RNA sample mediated by the optimized RNA-DNA hybridization among the viral RNA and four of the selected Au probes. As illustrated in FIG. 15A, the gold nanoparticles capped with ssDNA will be deposited on the surface of the sensor chip by the drop-casting method. To this end, the volume of Au-Px solution deposited on the surface has been optimized to maintain the integrity and avoid the swelling effect of the sensor platform. FIG. 23A depicts the SEM image of the sensor chip without the addition of any solution, FIG. 23B shows the surface after the addition of 10 μL of the solution, and FIG. 23C demonstrates the sensor surface after the addition of 12 μL of solution. As shown in FIG. 23C, FIG. 23D, FIG. 23E and FIG. 23F, the sensor maintains its integrity up to 10 μL of the solution, whereas adding 2 μL of extra solution led to obvious damage to the sensor surface and swelling in the paper fibers; the paper fibers become visible in the image. FIG. 24 depicts the SEM images of fibers in the bare filter paper. As shown in FIG. 23A, in the sensor chip, the paper's fibers are hidden underneath the graphene layer. As illustrated by FIG. 23B, the same holds true for the SEM image of the sensor chip after the addition of 10 μL of the solution where no obvious swelling signs, such as the appearance of thick fibers or distortion of the graphene configuration, were observed. On the other hand, FIGS. 23C to 23F show a clear sign of paper swelling where the paper fibers become obvious and thicker, causing damage in the sensor chip at the microscopic level. Therefore, 10 μL is the optimum maximum volume that can be added to the sensor chip while maintaining its integrity. An 8 μL amount is found to be sufficient to cover the entire sensor electrode and this volume has been adopted for making the sensors throughout this study, with a total number of gold nanoparticles of 24×10⁷ particles per sensor chip. A clinical sample of 2 μL volume has then been added to ensure the total volume added to the sensor chip did not exceed 10 μL.

It was expected that the ssDNA probes-capped AuNPs selectively bind with its target complementary SARS-CoV-2 RNA sequence and induce aggregation depending on their closely following target sequence positions. In the presence of SARS-CoV-2 RNA, the specific RNA-DNA hybridization led to the change in charge and electron mobility on the graphene surface, which brought the change in sensor output voltage. The change in the sensor electrical signal was observed instantaneously, which reached stability within a short duration of time with an average response time of less than 5 minutes, as shown in FIG. 6A. This illustrates a representative output signal of the sensor chip as a function of time after the addition of SARS-CoV-2 viral RNA. The sensor is composed of a filter paper as a base material that has been coated by graphene nanoplatelets to form a conductive film. The gold electrode has been deposited on the surface of the graphene film as the contact pad for the electrical readout. Finally, gold nanoparticles capped with ssDNA probes specific to the SARSCoV-2 RNA have been deposited on the sensor surface, which represents the recognition element of the sensor. In the presence of the SARS-CoV-2 RNA, the ssDNA mounted on the AuNPs' surface hybridized with its complementary target sequence on the surface of the graphene conductive film. The charges at the graphene-solution interface increase due to the hybridization of the viral RNA with the ssDNA sequence, which induces a variation in the graphene potential with a positive drift (eq 1 above). In order to eliminate the effect of the background noise, the change in the sensor output voltage was normalized to the sensor's initial voltage. The time-series signal was recorded as a response to the addition of the viral RNA sample consisting two main parts, transient and steady-state response. The addition of the RNA sample at a certain time point (t) represented a step input. The sensor exhibited a step response, which was represented by a sudden increase of the signal as a sharp peak (transient response). After some time, the sensor reached a steady-state response, which reflected the response to the viral RNA concentrations. This increase in the sensor signal after the addition of the sensor and following partial decrease can be seen in the whole electrochemical platform since it represents both the transient and the steady-state responses of the sensor. To further investigate the performance of the sensor, the dynamic response of the sensor, when deposited with Au-Pₘᵢₓ, was evaluated to various concentrations of viral RNA load extracted from Vero cells infected with the SARS-CoV-2 virus.

FIG. 6B illustrates the linear response (standard curve) of the electrochemical biosensor with increasing concentrations of viral RNA. This fully integrated device successfully exhibited a broad linear detection range from 585.4 copies/μL to 5.854×10⁷ copies/μL, with a sensitivity of 231 ((copies μL)⁻¹)⁻¹ for SARS-CoV-2 viral RNA. The sensor sensitivity is referred to as the change in the sensor output per unit change in the sensor input. The sensitivity, by definition, is the ratio of the measured output to the measured analyte or target and can be calculated as the slope of the system transfer function. In our case, the sensitivity is the ratio of the relative change in the sensor voltage (ΔV/V₀) to SARS-CoV-2 RNA concentration (copies/μL), which has units of (copies/μL)⁻¹ since ΔV/V₀ is unitless.

One of the important sensor performances is the limit of detection (LOD). The LOD represents the lowest amount of the target substrate that can be distinguished from its absence with a predefined confidence level, usually 99%. The limit of detection is usually calculated based on statistics from the blank measurement points and the slope (analytical sensitivity, as we mentioned earlier) of the sensor standard curve. The limit of detection of the sensor chip has thus been calculated based on LOD=3.3S_{XY}/slope and found to be 6.9 copies/μL. This significantly lower value of the LOD indicated the improved sensitivity and superior analytical performance of the designed device. However, as shown in FIG. 6C, the pristine graphene-based device and the citrate-stabilized AuNPs deposited on the graphene-based device did not show any significant output signal change in the presence of the viral RNA load. These results supported the importance of ssDNA probes and their sensitivity toward the target SARS-CoV-2 viral N gene.

### Example 20. Selectivity of the Electrochemical Biosensor Toward SARS-CoV-2

The selectivity of the SARS-CoV-2 biosensor from Examples 1 through 7 was tested in which the sensor output signal was recorded in the absence of SARS-CoV-2 viral RNA and in the presence of MERS-CoV and SARS-CoV viral RNA load. As shown in FIG. 6D, the sensor, when deposited with Au-Pₘᵢₓ, showed no significant change in the output signal toward either MERS-CoV or SARS-CoV RNA, demonstrating the selectivity of the device platform toward SARS-CoV-2 with no obvious cross-reactivity.

### Example 21. Selective Detection of SARS-CoV-2 in Clinical Samples

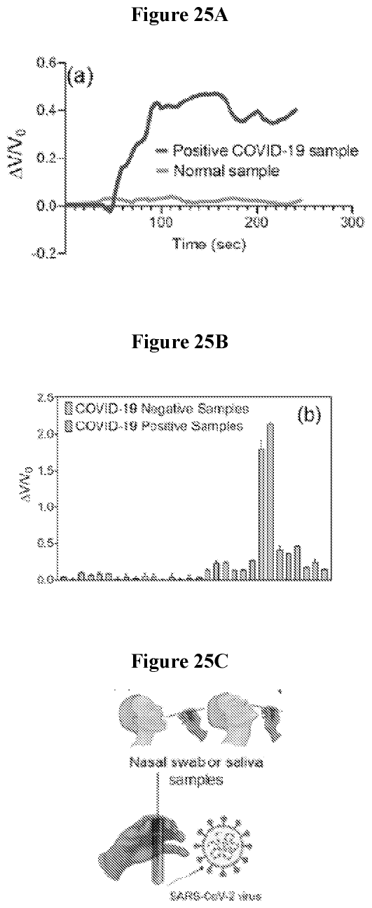

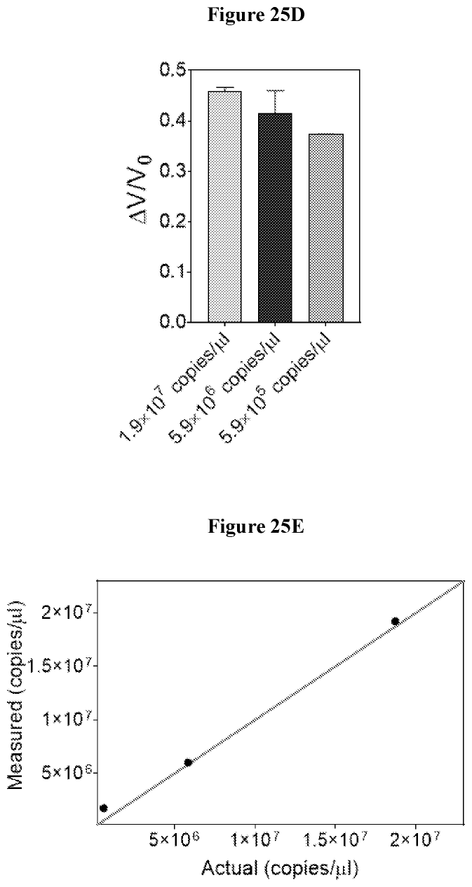

Finally, we evaluated the performance of the developed sensor chip in detecting the presence of SARS-CoV-2 RNA in clinical samples. The results are shown in FIG. 25A, FIG. 25B, FIG. 25C, FIG. 25D and FIG. 25E. Nasopharyngeal swab specimens were collected from COVID-19 patients and healthy asymptomatic subjects and stored in viral transfer medium (VTM) for future use, as shown in FIG. 25C. The results were benchmarked with a gold standard SARS-CoV-2 diagnostic kit known as LabGun COVID-19 RT-PCR kit (FDA approved). Prior to testing of patient samples, we investigated the sensor response toward samples that were collected from healthy asymptomatic subjects, confirmed using RT-PCR. FIG. 25A clearly shows that the sensor responds to the positive COVID-19 confirmed samples selectively and demonstrated a significant increase in sensor voltage compared to the negative samples. Thus, the sensor chip clearly discriminated between COVID-19 positive samples and healthy negative samples in a rapid manner. As shown in FIG. 25B, the sensor showed a negligible response toward the samples collected from a healthy individual, while a high relative change in the sensor chip voltage was observed for the positive COVID-19 confirmed clinical samples. Moreover, the sensor chip responded to COVID-19 positive samples with different viral RNA loads of 1.9×10⁷, 5.9×10⁶, and 5.9×10⁵, as shown in FIG. 25D.

Quantitative detection of the viral RNA load using a simple sensor chip is also important in monitoring the progression of SARS-CoV-2 infection after confirming the disease after the onset of symptoms. To this end, we evaluated the ability of the developed sensor chip to quantify the viral RNA load. As shown in FIG. 25E, the RNA concentration of the tested COVID-19 positive clinical samples (i.e., viral RNA load of 1.9×10⁷, 5.9×10⁶, and 5.9×10⁵) was estimated with high accuracy, where the RNA concentration of the clinical samples that were back-calculated from the standard curve was found to be highly correlated with the actual RNA concentration with Pearson's correlation of r=0.999.

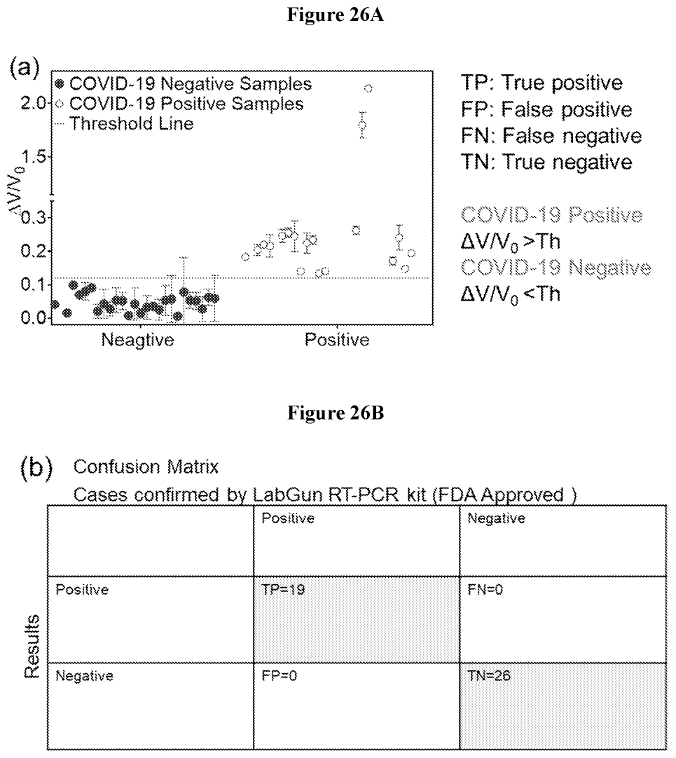

Finally, the sensor response toward the collected clinical samples has been used to distinguish the positive COVID-19 samples from the negative ones (i.e., by providing a “YES/NO” answer) using the thresholding process. The sensor response (i.e., the relative change in the sensor voltage (ΔV/V₀)) has been classified by applying a threshold value of 0.13, in which the samples giving a response higher than the threshold are considered as positive, whereas the samples with a response lower than 0.13 are assigned as negative. FIG. 26A illustrates the sensor chip response toward the 48 clinical samples; the samples that belong to the negative group all showed a response (ΔV/V₀) less than 0.13. On the other hand, the samples that showed a response higher than 0.13 belong to the COVID-19 positive group. Table 2 summarizes the amount of SARS-CoV-2 RNA in each clinical sample in copies/μL. FIG. 26B shows the confusion matrix, which summarized the performance of the sensor chip in identifying the COVID-19 positive and negative samples, by benchmarking the results to a gold-standard technique for SARS-CoV-2 diagnosis known as the LabGun COVID-19 RTPCR diagnosis kit (FDA EUA approved). As shown in FIG. 26B, the sensor was thus able to correctly assign the positive and negative samples to their respective groups with almost 100% accuracy, specificity, and sensitivity. It is worth mentioning that this threshold value is not universal and may be subject to change with an increasing number of tested clinical samples.

TABLE 2
The copy number of SARS-CoV-2 RNA in each
COVID-19 positive clinical sample tested.

| | RNA concentration |
|---|---|
| Sample Number | (Copies/μL) |
| 1 | 22057.56 |
| 2 | 33086.33 |
| 3 | 44115.11 |
| 4 | 55143.89 |
| 5 | 66172.67 |
| 6 | 77201.45 |
| 7 | 88230.23 |
| 8 | 99259 |
| 9 | 3196.992 |
| 10 | 4795.488 |
| 11 | 6393.984 |
| 12 | 7992.48 |
| 13 | 9590.976 |
| 14 | 11189.47 |
| 15 | 12787.97 |
| 16 | 14386.46 |
| 17 | 9015.625 |
| 18 | 38441.12 |
| 19 | 7011.323 |
| 20 | 15984.96 |
| 21 | 590000 |
| 22 | 5909000 |

### Example 22: Electrochemical Biosensor Comprising ASOs Having a Hairpin Loop Structure

An electrochemical biosensor was tested comprising the ASOs of Example 1, wherein the ASOs formed a hairpin loop structure. A first end of the ASOs was coupled with a sensing electrode, and a second end of the ASOs was conjugated to methylene blue.

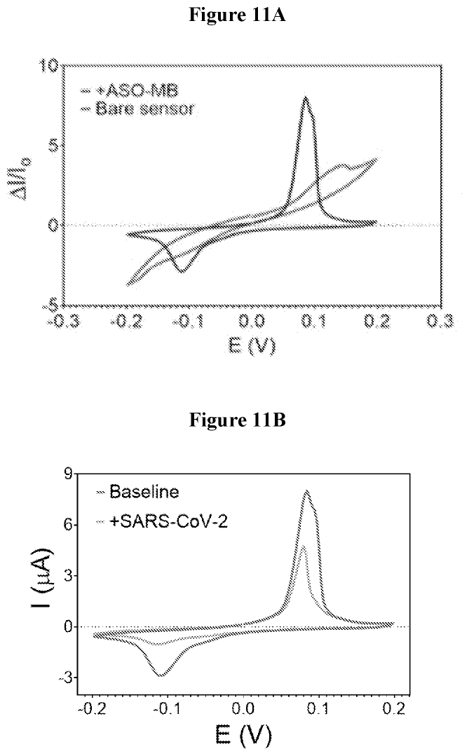

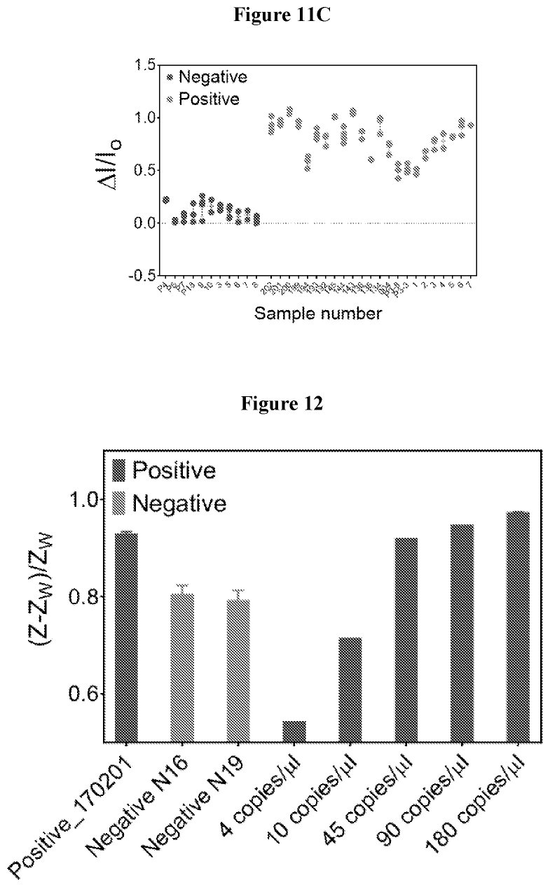

The successful modification of the surface of the sensing electrode with methylene blue conjugated ASO was confirmed by measuring cyclic voltammetry. FIG. 11A shows the baseline cyclic voltammetry curve observed before and after sensor conjugation with methylene blue conjugated ASOs in lysis buffer. Based on FIG. 11A, two peaks were observed in the cyclic voltammetry curve at the methylene blue redox potential. FIG. 11B depicts the change in current of a reference as compared to the change in current of a sample containing a biological pathogen of interest. In this case, the biological pathogen of interest was SARS-CoV-2, and the presence of SARS-CoV-2 was confirmed by RT-PCR. FIG. 11C illustrates the electrochemical biosensor response to SARS-CoV-2 positive and negative samples (such as confirmed using RT-PCR). As shown in FIG. 11C a significant change in the current was observed in the case of positive samples, whereas no significant change in the current was observed upon the addition of negative samples. This may be due to the increased distance between methylene blue and the sensor electrodes that results when the ASOs bind with the target sequence. The electrochemical biosensor was successful in discriminating positive samples from negative samples.

According to another exemplary embodiment, the sensor was responsive to approximately 22 copies/μl of the SARS-CoV-2 RNA when tested using a sample treated with lysis buffer. In another example, the limit of detection was calculated to be 286.33 copies/ml, as shown in FIG. 12. In still another example, the sensor also succeeds in differentiating positive samples from negative samples using RNA samples extracted from clinical samples with a limit of detection of 44 copies/μl, without any washing step, as shown in FIG. 12.

In certain embodiments, the electrochemical biosensor disclosed herein has 95% accuracy, 97% sensitivity, 93% specificity and a limit of detection of <2 copies/μl.

### Example 23. Adaptability of the Electrochemical Biosensor to Test for Other Pathogens

In order to adapt the protocols described in Examples 1 to 22 to detect other biological targets, the following steps could be used.

Suitable antisense oligonucleotide (ASO) sequences need to be designed that specifically target RNA of the disease-causing virus/pathogen. Particular attention should be paid to designing the ASO sequences since the target binding energies and binding disruption energies will influence the RNA-ASO hybridization process and sensitivity of the electrochemical biosensor.

Next, the inner, outer and loop primer sequences need to be altered to amplify the target gene sequence to which the ASOs will hybridize. Overall, by changing the sequences of ASOs and primers according to the principles described in the previously described Examples, the capability of the protocol could be expanded to other pathogens.

Suitable standardization of lysis buffers must be performed while utilizing this technology for the detection of other targets. The membranes of different microorganisms comprise varied biological materials and hence might behave differently in different types of lysis buffer. Therefore, to obtain an optimized result, one needs to standardize the lysis conditions for each target as was done during the development of this protocol for SARS-CoV-2.

Basic knowledge in molecular biology is required to design ASOs and NAA primers. Basic knowledge of material science, chemistry and electrochemical systems are also recommended to perform the methods demonstrated in the Examples described herein, including conjugating AuNPs or redox reporter molecules with ASOs and configuring the electrochemical sensor system. Once all the reagents and required materials are prepared, performing the test to detect a biological pathogen is quite straightforward. As the system, compositions and methods described herein offers the possibility of biological pathogen detection even without the extraction or amplification of RNA, it enables individuals with limited scientific expertise to conduct the test.

### Example 24. Preparation of ASOs for the Detection of Hepatitis C Virus

Following the methods set out in Example 1 and the principles of Example 23, ASOs were identified specific to Hepatitis C Virus (HCV). Specifically, two ASOs were selected, targeted towards (i) stem-loop structure within the 5′ noncoding region (5′-NCR) known to be important for internal ribosome entry site (IRES) function (ASO5) and (ii) sequences spanning the AUG used for initiation of HCV polyprotein translation (ASO6). The sequences of these ASOs are represented below in Table 3.

TABLE 3
Sequences of the HCV targeting ASOs

| | Sequence (5′-3′) |
|---|---|
| ASO5 | HS-C6-GCCTTTCGCGACCCAACACT (SEQ ID NO 10) |
| ASO6 | HS-C6-GTGCTCATGGTGCACGGTCT (SEQ ID NO 11) |

### Example 25. Detection of Hepatitis C Virus with Citrate-Stabilized Gold Nanoparticles and Electrochemical Biosensor

The ASOs identified in Example 24 are functionalized with gold nanoparticles and used to detect the presence of HCV in a sample.

Citrate-stabilized gold nanoparticles are synthesized in accordance with the method set out in Example 2. The ASOs identified in Example 24 are capped with the citrate-stabilized gold nanoparticles in accordance with the method set out in Example 3. An electrochemical biosensor with graphene nanoplatelets is fabricated in accordance with the method set out in Example 6.

The standard standardization and data collection protocols set out in Examples 7 and 8 are followed, and an electrical circuit is prepared in accordance with Example 10. The electrochemical biosensor and ASO-capped gold nanoparticles are characterized and optimized for the sensitive detection of HCV in accordance with the methods set out in Examples 13-19 and 21.

### Example 26. Detection of Hepatitis C Virus with Methylene Blue Conjugated ASOs

The ASOs identified in Example 24 are conjugated to methylene blue and functionalized to couple to a sensing electrode and used to detect the presence of HCV in a sample.

A first end of the ASOs is coupled with a sensing electrode, and a second end of the ASOs is conjugated to methylene blue in accordance with Example 22. Confirmation of successful modification of the surface of the sensing electrode with methylene blue conjugated ASOs and confirmation of detection of HCV is performed in accordance with the methods set out in Example 22.

### Example 27. Preparation of ASOs for the Detection of Influenza H1N1

Following the methods set out in Example 1 and the principles of Example 23, ASOs were identified specific to Influenza A H1N1. Specifically, two ASOs were selected, targeted towards the HA gene of Influenza A H1N1 (ASO 7 and ASO 8). The sequences of these ASOs are represented below in Table 4.

TABLE 4
Sequences of the Influenza A
H1N1 targeting ASOs

| | Target Sequence | ASO Sequence |
|---|---|---|
| | (5′-3′) | (5′-3′) |
| ASO7 | CUAGUACUGUGU | GACACTGTAGAC |
| | CUACAGUGUC | ACAGTACTAG |
| | (SEQ ID NO 12) | (SEQ ID NO 14) |
| ASO8 | ACAGGAAGCAA | CCCTGTGCTTT |
| | AGCACAGGG | GCTTCCTGT |
| | (SEQ ID NO 13) | (SEQ ID NO 15) |

### Example 28. Detection of Influenza H1N1 with Citrate-Stabilized Gold Nanoparticles and Electrochemical Biosensor

The ASOs identified in Example 27 are functionalized with gold nanoparticles and used to detect the presence of Influenza H1N1 in a sample.

Citrate-stabilized gold nanoparticles are synthesized in accordance with the method set out in Example 2. The ASOs identified in Example 26 are capped with the citrate-stabilized gold nanoparticles in accordance with the method set out in Example 3. An electrochemical biosensor with graphene nanoplatelets is fabricated in accordance with the method set out in Example 6.

The standard standardization and data collection protocols set out in Examples 7 and 8 are followed, and an electrical circuit is prepared in accordance with Example 10. The electrochemical biosensor and ASO-capped gold nanoparticles are characterized and optimized for the sensitive detection of Influenza H1N1 in accordance with the methods set out in Examples 13-19 and 21.

### Example 29. Detection of Influenza H1N1 with Methylene Blue Conjugated ASOs

The ASOs identified in Example 27 are conjugated to methylene blue and functionalized to couple to a sensing electrode and used to detect the presence of Influenza H1N1 in a sample.

A first end of the ASOs is coupled with a sensing electrode, and a second end of the ASOs is conjugated to methylene blue in accordance with Example 22. Confirmation of successful modification of the surface of the sensing electrode with methylene blue conjugated ASOs and confirmation of detection of Influenza H1N1 is performed in accordance with the methods set out in Example 22.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. An electrochemical biosensor for use in the detection of a biological pathogen in a sample, the electrochemical biosensor comprising:
a) a sensing element comprising a plurality of first anti-sense oligonucleotides, the sequence of which is complementary to a first nucleic acid sequence in a target gene of the biological pathogen;
b) a first electrode connected to a first end of each of the plurality of anti-sense oligonucleotides; and
c) a second electrode electrically connected to the first electrode;
wherein contact of the sample with the first electrode causes binding of the plurality of first anti-sense oligonucleotides to the first nucleic acid sequence in the target gene, to provide a signal to identify presence of the biological pathogen.

2. The electrochemical biosensor of claim 1, further comprising a plurality of second anti-sense oligonucleotides, the sequence of which is complementary to a second nucleic acid sequence in the target gene of the biological pathogen near to the first nucleic acid sequence;
wherein the first electrode is additionally connected to a first end of the plurality of second anti-sense oligonucleotides;
wherein the signal is provided additionally by binding of the plurality of second anti-sense oligonucleotides to the second nucleic acid sequence in the target gene.

3. The electrochemical biosensor of claim 1, wherein the biological pathogen is SARS-CoV-2, and wherein the sequence of the first anti-sense oligonucleotide comprises SEQ ID NO 6, SEQ ID NO 7, SEQ ID NO 8, or SEQ ID NO 9.

4. The electrochemical biosensor of claim 1, wherein the first anti-sense oligonucleotide has an unpaired probability for the first nucleic acid sequence of at least 0.5.

5. The electrochemical biosensor of claim 1, wherein the first anti-sense oligonucleotide has a binding energy of less than −8 kcal/mol.

6. The electrochemical biosensor of claim 1, wherein the first anti-sense oligonucleotide has a tendency to form a hairpin-loop structure.

7. The electrochemical biosensor of claim 1, wherein the second electrode is a counter electrode or a reference electrode.

8. The electrochemical biosensor of claim 1, further comprising a third electrode electrically connected to the first and second electrodes.

9. The electrochemical biosensor of claim 1, further comprising:
a) a substrate; and
b) a conductive film coated on the surface of the substrate;
wherein the first and second electrodes are deposited on the conductive film.

10. The electrochemical biosensor of claim 9, wherein the first anti-sense oligonucleotides are capped with conductive nanoparticles.

11. The electrochemical biosensor of claim 10, wherein the conductive nanoparticles are gold nanoparticles.

12. The electrochemical biosensor of claim 9, wherein the conductive film comprises graphene.

13. The electrochemical biosensor of claim 1, wherein the first anti-sense oligonucleotides form hairpin-loop structures in the absence of the target gene, and wherein the presence of the target gene in the sample causes the plurality of first anti-sense oligonucleotides to unfold and bind to the first nucleic acid sequences in the target gene, resulting in providing the signal.

14. The electrochemical biosensor of claim 13, further comprising a redox reporter molecule bound to the second end of each of the plurality of first anti-sense oligonucleotides, such that when the first anti-sense oligonucleotide forms a hairpin-loop structure, the redox reporter molecule is brought within proximity of the first electrode, and wherein in the presence of the target gene the first anti-sense oligonucleotide unfolds moving the redox reporter molecule away from the first electrode, resulting in providing the signal.

15. The electrochemical biosensor of claim 14, wherein the redox reporter molecule is methylene blue.

16. A method of detecting a biological pathogen in a sample, the method comprising:
a) providing a plurality of first anti-sense oligonucleotides having a sequence complementary to a first nucleic acid sequence in a target gene of the biological pathogen;
b) providing a first electrode and a second electrode electrically connected to one another;
c) connecting a first end of the pluralities of the first anti-sense oligonucleotides to the first electrode; and
d) contacting the first electrode to the sample;
e) measuring a signal from the first and second electrodes;
wherein the binding of the target gene of the biological pathogen to the plurality of first anti-sense oligonucleotides provides the signal identifying the presence of the biological pathogen in the sample.

17. The method of claim 16, further comprising:
a) providing a plurality of second anti-sense oligonucleotides, the sequence of which is complementary to a second nucleic acid sequence in the target gene of the biological pathogen near to the second nucleic acid sequence; and
b) connecting a first end of the pluralities of the second anti-sense oligonucleotides to the first electrode;
wherein the signal is provided additionally by binding of the target gene of the biological pathogen to the plurality of second anti-sense oligonucleotides.

18. A method of selecting at least one anti-sense oligonucleotide probe for use in detection of a biological pathogen, comprising:
a) identifying a target gene in the biological pathogen;
b) obtaining the nucleic acid sequence of the target gene;
c) producing a library of anti-sense oligonucleotides of a length of about 20 nucleotides, wherein the sequence of each anti-sense oligonucleotide is complementary to a section of the nucleic acid sequence in the target gene and wherein:
i. guanine and cysteine form from 40 to 60 percent of each anti-sense oligonucleotide in the library;
ii. none of the anti-sense oligonucleotides in the library are complementary to a section of the target gene with the sequence GGGG;
iii. the average unpaired probability of each of the anti-sense oligonucleotides in the library is at least 0.5;
d) ranking the anti-sense oligonucleotides in the library in descending order of average unpaired probability; and
e) selecting the at least one anti-sense oligonucleotide probe from the anti-sense oligonucleotides in the library, wherein the binding energy of the at least one anti-sense oligonucleotide probe is less than −8 kcal/mol, compared with the nucleic acid sequence of the target gene.

19. The method of claim 18, wherein the at least one anti-sense oligonucleotide probe is additionally selected based on comparative binding disruption energies and binding energies with the nucleic acid sequence of the target gene.

20. The method of claim 18, wherein the at least one anti-sense oligonucleotide probe is additionally selected for a tendency to form a hairpin-loop structure.


SEQUENCE LISTING (15 sequences)

SEQ ID NO: 1 - DNA, 1380 bp, SARS-CoV-2 virus
  misc_feature: Target N gene sequence of SARS-CoV-2 isolate 2019-nCoV/USA-WA1-A12/2020
cgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgc

SEQ ID NO: 2 - RNA, 20 nt, SARS-CoV-2 virus
acaccaaaagaucacauugg

SEQ ID NO: 3 - RNA, 20 nt, SARS-CoV-2 virus
cccgcaauccugcuaacaau

SEQ ID NO: 4 - RNA, 20 nt, SARS-CoV-2 virus
cagaacaaacccaaggaaau

SEQ ID NO: 5 - RNA, 20 nt, SARS-CoV-2 virus
acugauuacaaacauuggcc

SEQ ID NO: 6 - DNA, 20 bp, Artificial Sequence
  Antisense oligonucleotide 1 (ASO1)
ccaatgtgatcttttggtgt

SEQ ID NO: 7 - DNA, 20 bp, Artificial Sequence
  Antisense oligonucleotide 2 (ASO2)
attgttagcaggattgcggg

SEQ ID NO: 8 - DNA, 20 bp, Artificial Sequence
  Antisense oligonucleotide 3 (ASO3)
atttccttgggtttgttctg

SEQ ID NO: 9 - DNA, 20 bp, Artificial Sequence
  Antisense oligonucleotide 4 (ASO4)
ggccaatgtttgtaatcagt

SEQ ID NO: 10 - DNA, 20 bp, Artificial Sequence
  Antisense oligonucleotide 5 (ASO5)
gcctttcgcgacccaacact

SEQ ID NO: 11 - DNA, 20 bp, Artificial Sequence
  Antisense oligonucleotide  (ASO6)
gtgctcatggtgcacggtct

SEQ ID NO: 12 - RNA, 22 nt, Influenza A H1N1 virus
cuaguacugugucuacaguguc

SEQ ID NO: 13 - RNA, 20 nt, Influenza A H1N1 virus
acaggaagcaaagcacaggg

SEQ ID NO: 14 - DNA, 22 bp, Artificial Sequence
  Antisense oligonucleotide 7 (ASO7)
gacactgtagacacagtactag

SEQ ID NO: 15 - DNA, 20 bp, Artificial Sequence
  Antisense oligonucleotide 8 (ASO8)
ccctgtgctttgcttcctgt